(19) United States
(12) Patent Application Publication
(10) Pub. No.: US 2022/0002435 A1
(43) Pub. Date: Jan. 6, 2022

(54) ANTIBODIES TARGETING EPN1

(71) Applicant: IMMUNOME, INC., Exton, PA (US)

(72) Inventor: Matthew K. ROBINSON, Blue Bell, PA (US)

(21) Appl. No.: 17/281,663

(22) PCT Filed: Oct. 2, 2019
(86) PCT No.: PCT/US2019/054259

§ 371 Date: Mar. 31, 2021

Related U.S. Application Data
(60) Provisional application No. 62/740,092, filed on Oct. 2, 2018.

(51) Int. Cl. C07K 16/30; C07K 16/46; C12N 15/63

(52) U.S. Cl. CPC C07K 16/30; C07K 16/468; C12N 15/63; C07K 2317/622; C07K 2317/34; C07K 2317/31; C07K 2317/73; C07K 2317/33

(57) ABSTRACT

Described herein, are antibodies specific for epsin-1 (EPN1), an antigen expressed on the surface of various cancer cells. Methods for treating, ameliorating and diagnosing various types of cancers characterized by EPN1-expressing tumor cells are also disclosed.

### FIELD OF THE INVENTION

The field of this invention relates to antibody-based therapeutics for the treatment or detection of cancer.

### BACKGROUND

The human adaptive immune system responds through both cellular (T cell) and humoral (B cell) processes. The humoral response results in selection and clonal amplification of B cells that express surface bound immunoglobulin (Ig) molecules capable of binding to antigens. The processes of somatic hypermutation and class switching take place concordant with the clonal amplification. Together these processes lead to secreted antibodies that have been affinity matured against a target antigen and contain a constant domain belonging to one of the four general classes (M, D, A, G, or E). Each class of antibody (IgM, IgD, IgA, IgG, and IgE) interact in distinct ways with the cellular immune system. Hallmarks of antibodies that have been affinity matured against a target antigen can include: 1) nucleotide, and subsequent amino acid, changes relative to the germline gene, 2) high binding affinity for the target antigen, 3) binding selectivity for the target antigen as compared to other proteins.

It is well understood that oncology patients can mount an immune response against tumor cell antigens. Those antigens can result either from genetic changes within the tumor that lead to mutated proteins or aberrant presentation, of otherwise normal, proteins to the immune system. Aberrant presentation may occur through processes that include, but are not limited to, ectopic expression of neonatal proteins, mis-localization of intracellular proteins to the cell surface, or lysis of cells. Aberrant expression of enzymes that lead to changes in glycosylation of proteins can also result in generation of non-self antigens that are recognized by the humoral immune system.

Antibodies, which bind selectively to disease-related proteins, including those related to cancer, have proven successful at modulating the functions of their target proteins in ways that lead to therapeutic efficacy. The ability of the human immune system to mount antibody responses against mutated, or otherwise aberrant, proteins suggests that patients' immune responses may include antibodies that are capable of recognizing, and modulating the function of, critical tumor-drivers.

Membrane trafficking contributes to the regulation of a wide range of cellular processes. Internalization of cell surface receptors is a critical mechanism for appropriate modulation of growth factor receptor mediated signaling. Internalization via clathrin-coated vesicles represents one pathway for internalization of cancer relevant receptors from the cell surface. Loading of those receptors into clathrin-coated pits (CCPs), for subsequent internalization in clathrin-coated vesicles, is one of the first steps in the pathway. Loading of receptors into CCPs is dictated in part by interaction with adaptor molecules, such as Epsin-1 (EPN1).

EPN1 is an approximately 60.3 kDa protein that localizes to cellular membranes. It contains a PI(4,5)P2-, ubiquitin-, and clathrin/AP-2-interacting domains. Knocking down expression of endogenous expression of EPN1, overexpressing mutant forms of EPN1, or treating cells with agents designed to block interaction of EPN1 with its cargo molecules can inhibit internalization of known CCP-dependent cargo. Examples of such a cargo are VEGFR and ERBB3.

### SUMMARY OF THE INVENTION

The invention provides antibodies specific for the protein, Epsin 1 (EPN1), including EPN1-specific antibodies with V_{H} and V_{L} framework and complementary regions correlating with SEQ ID NOS: 4 and 8, respectively, or fragments thereof, as well as EPN1-specific antibodies with one or more complementarity-determining regions (“CDRs”) H-CDR1, H CDR2, H-CDR3, L-CDR1, L CDR2, and L-CDR3 corresponding to SEQ ID NOS: 9-14, respectively.

Compositions and methods for treating, ameliorating or diagnosing various types of cancers characterized by EPN1-expressing tumor cells are also provided by the invention. Embodiments of the foregoing compositions and methods include naturally-occurring anti-EPN1 antibodies, fragments thereof, variants thereof. For example, embodiments of antibodies of the invention include, but are not limited to single-domain antibodies, Fab fragments, Fab′ fragments, F(ab)′₂ fragments, single chain Fv proteins (“scFv”), and disulfide stabilized Fv proteins (“dsFv”). In various embodiments, antibodies of the invention can contain amino acid sequence modifications that preserve residues necessary for correct folding and stabilization between the V_{H} and the V_{L} regions, as well as preserve the low pl and low toxicity of the molecules. Antibodies used in methods of treatment or diagnosis, according to the invention, can, in certain embodiments be conjugated to effector molecules, including therapeutic agents, diagnostic agents, or half-life and bioavailability-enhancing molecules.

Antibodies according to the invention can be produced by various recombinant expression systems. Such systems include host-expression vector systems may be utilized to express an antibody according to the invention, by transforming or transfecting the cells with an appropriate nucleotide coding sequences for an antibody according to the invention. In various embodiments, a host expression cell system can modulate expression of inserted sequences coding for an antibody according to the invention, or modify and process the antibody gene product as desired.

As stated above, anti-EPN1 antibodies of the invention can be used in compositions and methods for preventing, treating, or ameliorating cancer in a subject, such as, for example, lung cancer or melanoma. Accordingly, in various embodiments of the invention, a therapeutically effective amount of an antibody is administered to a subject in an amount sufficient to inhibit growth, replication or metastasis of cancer cells, or to inhibit a sign or a symptom of the cancer. In these embodiments, the antibodies are formulated into compositions that are suitable for the mode of administration of the composition to the subject.

With respect to compositions and methods for using antibodies of the invention for diagnosing or monitoring the presence of cancer in a subject, the detection of cancer can be performed in vitro in certain embodiments, or in vivo in other embodiments. An embodiment of the invention can also be a kit for detecting EPN1 positive cells. Such a kit will typically contain an antibody composition according to the invention, buffers and reagents for the particular application for which the kit is designed, and instructional materials.

### BRIEF DESCRIPTION OF THE FIGURES

FIG. 1 is a photomicrograph showing the detection of the hybridoma-produced anti-EPN1 antibody PR045-2H11 bound to a pool of live, intact cancer cell lines. Fluorophore-labelled goat anti-human IgG secondary antibodies were used in combination with a LI-COR Odyssey® Sa imaging system to detect binding of binding of PR045-2H11-produced antibodies.

FIG. 2 shows a quantitative analysis of the fluorescent signals observed in a subset of the wells depicted in FIG. 1. Solid black bars represent potential screening hits, speckled bars represent background signal, white open bars represent positive control wells.

FIG. 3 shows the concentration-dependent binding curve observed for IMM20059 binding to intact A549 lung cancer cell lines by flow cytometry with an Attune™ N×T instrument (Life Technologies). Binding of IMM20059 to intact cells was detected with fluorophore-labelled anti-human secondary antibodies.

FIG. 4 shows the concentration-dependent binding curve observed for IMM20059 binding to intact Huh7 hepatocellular carcinoma cells by flow cytometry with an Attune™ N×T instrument (Life Technologies). Binding of IMM20059 to intact cells was detected with fluorophore-labelled anti-human secondary Abs.

FIG. 5 shows a scanned image of an SDS-PAGE resolution of the immunoprecipitation by IMM20059 of a 65 kDa protein under both stringent (200 mM and 300 mM NaCl) wash conditions, and under no wash conditions.

FIG. 6 shows quantitative dot blot results depicting selectivity of IMM20059 for EPN1 over its homolog EPN2. Binding of IMM20059 was analyzed by dot blot against increasing concentrations of recombinant EPN1 or EPN2.

FIG. 7 shows a flow cytometry analysis of parental HEK293 and an EPN1−/− variant of HEK293 generated by CRISPR-based knock-out. Fixed and permeabilized cells were stained with either IMM20059 or a commercial anti-EPN1 mAb. Binding was detected with a fluorophore-labelled anti-human secondary antibody.

FIG. 8 is a photomicrograph showing the immunofluoresence staining pattern observed for IMM20059 against the H460 human lung cancer cell line. Binding was detected with a fluorophore-labelled anti-human secondary antibody.

FIG. 9 is a photomicrograph showing the immunofluoresence staining pattern observed for an anti-EPN1 monoclonal antibody against the H460 human lung cancer cell line. Binding was detected with a fluorophore-labelled anti-mouse secondary antibody.

FIG. 10 is a PyMOL-derived representation of the Epsin N-terminal Homology (ENTH) domain of the rat EPN1 molecule (RCSB PDB: 1edu), which is 100% conserved relative to human EPN1 at the amino acid sequence level. Residues in dark gray cartoon comprise the proteolytically-derived peptides identified as crosslinking to IMM20059. Residues in light gray ribbon are outside of the crosslinked peptides. Residues in spheres represent the amino acids that were crosslinked directly to IMM20059.

FIG. 11 is a PyMOL-derived representation of the Epsin N-terminal Homology (ENTH) domain of the rat EPN1 molecule (RCSB PDB: 1edu), which is 100% conserved to human EPN1 at the amino acid level. Residues in dark gray cartoon represent the proteolytically-derived peptides identified as crosslinking to IMM20059. Residues in light gray ribbon are outside of the crosslinked peptides. Residues in spheres represent amino acids that differ between human EPN1 and human EPN2. Their position within the IMM20059 binding site provide rationale for specificity toward EPN1 vs EPN2.

FIG. 12 is a PyMOL-derived representation of the Epsin N-terminal Homology (ENTH) domain of the rat EPN1 molecule (RCSB PDB: 1edu), which is 100% conserved to human EPN1 at the amino acid level. Residues in dark gray cartoon comprise the proteolytically-derived peptides identified as crosslinking to IMM20059. Residues in light gray ribbon are outside of the crosslinked peptides. Residues in spheres represent amino acids that differ between human EPN1 and human EPN3. Their position within the IMM20059 binding site provide rationale for specificity toward EPN1 vs EPN3.

FIG. 13 is a multisequence alignment of human, rat, and mouse EPN1 amino acid sequences. The underlined region of the ENTH domain in human EPN1 corresponds to the amino acid peptides that compose the IMM20059 binding site. Murine and rat EPN1 are 100% identical to human EPN1 within this region, providing rationale for cross-reactivity to murine EPN1.

FIG. 14 is a multisequence alignment of human EPN1, EPN2, and EPN3 amino acid sequences. Human EPN1 is 56.7% and 49.6% identical to human EPN2 and EPN3, respectively.

FIG. 15 shows a flow cytometry analysis demonstrating that IMM20059 cross-reacts with murine EPN1 antigen. Surface and intracellular staining of cells of the murine NIH3T3 and human MFE296 cell lines was performed. Cell surface and intracellular binding of IMM20059 was observed in pools of both cell lines. A commercially available anti-EPN1 antibody known to cross-react with both mouse and human EPN1 bound similarly to NIH3T3 and MFE296 cells. However, the commercial antibody failed to interact with EPN1 at the cell surface in both pools of cells.

FIG. 16 shows the results of a cell proliferation assay, demonstrating that EPN1−/− variants of HEK293 proliferate more slowly than the parental HEK293 cells.

FIG. 17 is a graph depicting the tumor growth observed with B16F0 melanoma tumor model grown in C57Bl/6 mice. Cohorts of animals were treated weekly via intraperitoneal injection (IP) with isotype control (dashed line, black circles), anti-CTLA4 (dotted line, black square), or IMM20059 (solid line, black triangle) at 10 mg/kg.

### DETAILED DESCRIPTION

The invention described herein is directed to compositions and methods related to antibodies, which contain an amino acid sequence corresponding to SEQ ID NO: 4, or a portion thereof, and SEQ ID NO: 8, or a portion thereof. Indeed, an antibody according to the invention may include: an amino acid sequence that shares at least 90% 91%, 92%, 93%, 94%, 95%, 96%, 97%, 98%, 99%, or 100% sequence identity with an amino acid sequence corresponding to SEQ ID NO: 4, or a portion thereof; an amino acid sequence that shares at least at least 90%, 91%, 92%, 93%, 94%, 95%, 96%, 97%, 98%, 99%, or 100% sequence identity with an amino acid sequence corresponding to SEQ ID NO: 8, or a portion thereof; or both.

As used herein, the term “sequence identity” refers to the similarity between two, or more, amino acid or nucleic acid sequences. Sequence identity is typically measured in terms of percentage identity (or similarity or homology); the higher the percentage, the more similar the two sequences are. In addition to containing an amino acid sequence corresponding with SEQ ID NO: 4 or 8, an antibody according to the invention also specifically binds to EPN1, an antigen expressed on the surface of various carcinoma cells, including carcinoma cells of epithelial origin. Therefore, antibodies described herein can be included in compositions, which are useful for methods of diagnosing or treating various types of cancers characterized by EPN1-expressing tumor cells.

With respect to structure, an “antibody” according to the invention refers to a polypeptide ligand composed of, at least, a light chain or heavy chain immunoglobulin variable region that specifically binds an epitope of an antigen. For example, an antibody may be an immunoglobulin molecule composed of a heavy and a light chain, each of which has a variable region, respectively, termed the variable heavy (“V_{H}”) region and the variable light (“V_{L}”) region. Together, a V_{H} region and a V_{L} region form a fragment variable “Fv” that is responsible for the specific binding of the antibody to its antigen.

An antibody according to the invention may be an intact immunoglobulin, or a variant of an immunoglobulin, or a portion of an immunoglobuilin. A naturally occurring immunoglobulin has two heavy (H) chains and two light (L) chains, each of which, contains a constant region and a variable region, and are interconnected by disulfide bonds. There are two types of light chains, which are termed lambda (“λ”) and kappa (“κ”). There are five main heavy chain classes, also known as isotypes, which determine functional activity of an antibody molecule: IgM, IgD, IgG, IgA and IgE. In addition to its variable domain, a heavy chain also has three constant domains (CH1, CH2, CH3). The constant regions of the Abs may mediate the binding of the immunoglobulin to host tissues or factors, including various cells of the immune system (e.g., effector cells) and the first component (C1q) of the classical complement system.

Light and heavy chain variable regions contain “framework” regions interrupted by three hypervariable regions, called complementarity-determining regions (“CDRs”). The CDRs are primarily responsible for binding to an epitope of an antigen. The sequences of the framework regions of different light or heavy chains are relatively conserved within a species, and serve to position and align the CDRs in three-dimensional space. The three CDRs of each chain are typically referred to as CDR1, CDR2, and CDR3, numbered sequentially starting from the N-terminus, and are often identified by the chain in which the particular CDR is located. Accordingly, heavy chain CDRs are designated H-CDR1, H-CDR2, and H-CDR3; likewise, light chain CDRs are designated L-CDR1, L-CDR2, and L-CDR3. An antigen-binding fragment, one constant and one variable domain of each of the heavy and the light chain is referred to as an Fab fragment. An F(ab)′₂ fragment contains two Fab fragments, and can be generated by cleaving an immunoglobulin molecule below its hinge region.

Amino acid sequences of V_{H} and V_{L} framework and complementary regions of an antibody according to the invention correlate with SEQ ID NOS: 4 and 8, respectively. More particularly, based on the ImMunoGeneTics database (“IMGT”) numbering system, (Lefranc, M.-P. et al., Nucleic Acids Research, 27, 209-212 (1999)), H-CDR1, H-CDR2, and H-CDR3 correspond to residues 26-33 (SEQ ID NO: 9), 51-58 (SEQ ID NO: 10), and 97-113 (SEQ ID NO: 11) of SEQ ID NO: 4. The analagous L-CDR1, L-CDR2, and L-CDR3 amino acid sequences of an antibody according to the invention correspond to residues 27-32 (SEQ ID NO: 12), 50-56 (SEQ ID NO: 13), and 89-96 (SEQ ID NO: 14) of SEQ ID NO: 8. An antibody according to the invention contains at least one of the foregoing CDR sequences; therefore, the combination of CDRs of an antibody may be, for example: (H-CDR1 and L-CDR1); (H-CDR2 and L-CDR2); (H-CDR3 and L-CDR3); (H-CDR1, L-CDR1, H-CDR2 and L-CDR2); (H-CDR1, L-CDR1, H-CDR3 and L-CDR3); (H-CDR2, L-CDR2, H-CDR3 and L-CDR3); or (H-CDR1, L-CDR1, H-CDR2, L-CDR2, H-CDR3 and L-CDR3).

Antibodies according to the invention are monoclonal antibodies, meaning an antibody is produced by a single clonal B-lymphocyte population, a clonal hybridoma cell population, or a clonal population of cells which have been transected with the genes of a single antibody, or portions thereof. Monoclonal antibodies are produced by methods known to those of skill in the art, for instance by making hybrid antibody-forming cells from a fusion of myeloma cells with immune lymphocyte cells.

Monoclonal antibodies according to the invention are also typically humanized monoclonal antibodies. More specifically, a “human” antibody, also called a “fully human” antibody, according to the invention, is an antibody that includes human framework regions and CDRs from a human immunoglobulin. For example, the framework and the CDRs of an antibody are from the same originating human heavy chain, or human light chain amino acid sequence, or both. Alternatively, the framework regions may originate from one human antibody, and be engineered to include CDRs from a different human antibody.

An antibody according to the invention may also be an immunoglobulin fragment. Examples of immunoglobulin variants that are considered antibodies according to the invention include single-domain antibodies (such as VH domain antibodies), Fab fragments, Fab′ fragments, F(ab)′₂ fragments, single chain Fv proteins (“scFv”), and disulfide stabilized Fv proteins (“dsFv”). A VH single-domain antibody is an immunoglobulin fragment consisting of a heavy chain variable domain. An Fab fragment contains a monovalent antigen-binding immunoglobulin fragment, which can be produced by digestion of whole antibody with the enzyme papain to yield an intact light chain and a portion of one heavy chain. Similarly, an Fab′ fragment also contains a monovalent antigen-binding immunoglobulin fragment, which can be produced by digestion of whole antibody with the enzyme pepsin, followed by reduction, to yield an intact light chain and a portion of the heavy chain. Two Fab′ fragments are obtained per immunoglobulin molecule. An (Fab′)₂ fragment is a dimer of two Fab′ fragments, that can be obtained by treating whole antibody with the enzyme pepsin without subsequent reduction, so Fab′ monomers remain held together by two disulfide bonds. An Fv fragment is a genetically engineered fragment containing the variable region of the light chain and the variable region of the heavy chain expressed as two chains. A single chain (“sc”) antibody, such as scFv fragment, is a genetically engineered molecule containing the V_{L} region of a light chain, the V_{H} region of a heavy chain, linked by a suitable polypeptide linker as a genetically fused single chain molecule. A dimer of a single chain antibody, such as a scFV₂ antibody, is a dimer of a scFV, and may also be known as a “miniantibody”. A dsFvs variant also contains a V_{L} region of an immunoglobulin and a V_{H} region, but the chains have been mutated to introduce a disulfide bond to stabilize the association of the chains.

One of skill in the art will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pl and low toxicity of the molecules. Amino acid substitutions such as, at most one, at most two, at most three, at most four, or at most, five amino acid substitutions can be made in the V_{H} and the V_{L} regions to increase yield. Conservative amino acid substitution tables providing functionally similar amino acids are well known to one of ordinary skill in the art. The following six groupings of amino acids are examples of amino acids that are considered to be conservative substitutions for one another: i) Alanine (A), Serine (S), and Threonine (T); ii) Aspartic acid (D) and Glutamic acid (E); iii) Asparagine (N) and Glutamine (Q); iv) Arginine (R) and Lysine (K); v) Isoleucine (I), Leucine (L), Methionine (M), and Valine (V); and vi) Phenylalanine (F), Tyrosine (Y), and Tryptophan (W).

An antibody according to the invention may also include a “tagged” immunoglobulin CH3 domain to facilitate detection of the biologic against a background of endogenous antibodies. More particularly, a tagged CH3 domain is a heterogenous antibody epitope that has been incorporated into one or more of the AB, EF, or CD structural loops of a human IgG-derived CH3 domain. CH3 tags are preferably incorporated into the structural context of an IgG1 subclass antibody, other human IgG subclasses, including IgG2, IgG3, and IgG4, are also available according to the invention. Epitope-tagged CH3 domains, also referred to as “CH3 scaffolds” can be incorporated into any antibody of the invention having a heavy chain constant region, generally in the form of an immunoglobulin Fc portion. Examples of CH3 scaffold tags, and methods for incorporating them into antibodies are disclosed in PCT Patent Application No. PCT/US19/32780. Antibodies used to detect epitope tagged CH3 scaffolds are generally referred to herein as “detector antibodies”.

Therapeutic and diagnostic effectiveness of an antibody according to the invention correlates with its binding affinity for its target antigen. Binding affinity may be calculated by a modification of the Scatchard method described by Frankel et al., Mol. Immunol., 16:101-106, 1979. Alternatively, binding affinity may be measured by the dissociation rate of an antibody from its antigen. Various other methods can also be used to measure binding affinity, including, for example, surface plasmon resonance (SPR), competition radioimmunoassay, ELISA, and flow cytometry.

An antibody that “specifically binds” an antigen is an antibody that binds the antigen with high affinity and does not significantly bind other unrelated antigens. High affinity binding of an antibody to its antigen is mediated by the binding interaction of one or more of the antibody's CDRs to an epitope, also known as an antigenic determinant, of the antigen target. Epitopes are particular chemical groups or peptide sequences on a molecule that are antigenic, meaning they are capable of eliciting a specific immune response. An epitope that is specifically bound by an antibody according to the invention, may be, for example, contained within a protein expressed by cells of one or more types of cancer. In general, an antibody exhibits “high affinity binding” if its dissociation constant value (“K_{D}”) is 50 nM, or less. Therefore, an antibody according to the invention exhibits high affinity binding if the K_{D} between the antibody and Epsin 1 (“EPN1”), or a portion thereof that contains an epitope of the antibody according to the invention, is 50 nM or less. For example, an antibody according to the invention exhibits high affinity binding to EPN1 if the K_{D} value is 40 nM or less, 30 nM or less, 20 nM or less, 10 nM or less, 9 nM or less, 8 nM or less, 7 nM or less, 6 nM or less, 5 nM or less, 4 nM or less, 3 nM or less, 2 nM or less, or 1 nM or less.

High affinity binding of an antibody according to the invention can, for example, be described with respect to its binding to a cell that expresses EPN1. More particularly, an antibody according to the invention exhibits high affinity binding to EPN1-expressing cells if it exhibits a half maximal effective concentration (EC₅₀) value of 10 nM or less, 9 nM or less, 8 nM or less, 7 nM or less, 6 nM or less, 5 nM or less, 4 nM or less, 3 nM or less, 2 nM or less, or 1 nM or less.

Therapeutic and diagnostic uses of antibodies according to the invention may include uses of immunoconjugates. As described herein, an immunoconjugate is a chimeric molecule, which comprises an effector molecule linked to an antibody according to the invention. As referred to herein, an effector molecule is the portion of an immunoconjugate that is intended to have a desired effect on a cell to which the immunoconjugate is targeted, or an effector molecule may serve to increase the half-life or bioavailability of an antibody according to the invention. General examples of effector molecules include therapeutic agents, (such as toxins and chemotherapeutic drugs), diagnostic agents, (such as detectable markers), and half-life and bioavailability-enhancing molecules, (such as lipids).

Effector molecules can be linked to an antibody according to the invention using any number of means known to those of skill in the art, including covalent and noncovalent attachment means. The procedure for attaching an effector molecule to an antibody may vary according to the chemical structure of the effector. Polypeptides typically contain a variety of functional groups, such as a carboxylic acid (COOH) group, a free amine (—NH₂), and a sulfhydryl (SH) group, that are available for reaction with a suitable functional group on an antibody to result in the binding of the effector molecule. Alternatively, an antibody according to the invention can be derivatized to expose or attach additional reactive functional groups. Derivatization may involve attachment of any of a number of known linker molecules. A linker can be any molecule used to join the antibody to the effector molecule. For example, a linker can form covalent bonds to both the antibody and to the effector molecule. Suitable linkers are well known to those of skill in the art and include, but are not limited to, straight or branched-chain carbon linkers, heterocyclic carbon linkers, or peptide linkers. Where the effector molecule are polypeptides, the linkers may be joined to the constituent amino acids through their side groups, such as through a disulfide linkage to cysteine, or to the alpha carbon amino and carboxyl groups of the terminal amino acids. Recombinant technology may be used to make two or more polypeptides, including linker peptides, into one contiguous polypeptide molecule.

Therapeutic agents that can be conjugated to an antibody according to the invention include, but are not limited to, nucleic acids, proteins, peptides, amino acids or derivatives, glycoproteins, radioisotopes, lipids, carbohydrates, recombinant viruses, or small-molecule drugs. Nucleic acid therapeutic and diagnostic moieties include antisense nucleic acids, derivatized oligonucleotides for covalent cross-linking with single or duplex DNA, and triplex forming oligonucleotides.

Therapeutic agents can also be chemotherapeutic agents in certain embodiments of the invention. As referred to herein, a chemotherapeutic agent is any chemical agent, including radioactive agents, with therapeutic usefulness in the treatment of diseases characterized by abnormal cell growth. Such diseases include tumors, neoplasms, and cancer as well as diseases characterized by hyperplastic growth. For example an immunoconjugate according to the invention may be administered to a subject as part of a regimen for treating at least one type of cancer, or other hyperplastic disorder. A chemotherapeutic molecule may be directly conjugated to an antibody according to the invention, or it may be included in a linked encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are well known to those of skill in the art (see, for example, U.S. Pat. No. 4,957,735; and Connor et al., Pharm Ther 28:341-365, 1985).

As stated above, therapeutic agents according to the invention may also be toxins. Examples of toxins that may be linked to an antibody according to the invention include, but are not limited to, abrin, ricin, Pseudomonas exotoxin (“PE”, such as PE35, PE37, PE38, and PE40), diphtheria toxin (“DT”), botulinum toxin, saporin, restrictocin, gelonin, bouganin, and modified toxins thereof. In general though, a toxin in the context of the invention is a molecule that is toxic to a cell.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of a linker to release the effector molecule from an antibody according to the invention may be prompted by enzymatic activity or conditions to which the immunoconjugate is subjected either inside the target cell or in the vicinity of the target site. Alternatively, after specifically binding its target antigen, an antibody according to the invention can be internalized by the cell expressing the target antigen.

Therapeutic antibodies according to the invention, including therapeutic immunoconjugates, can be used in methods for preventing, treating, or ameliorating a disease in a subject. More particularly, therapeutic antibodies according to the invention can be used for preventing, treating, or ameliorating cancer, for example, lung cancer or melanoma. “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number of size of metastases. “Ameliorating” refers to the reduction in the number or severity of signs or symptoms of a disease, such as cancer. A method for preventing, treating, or ameliorating cancer may require the administration of a composition, comprising an effective amount of an antibody according to the invention, to a subject to inhibit tumor growth or metastasis, comprising selecting a subject with a cancer that expresses the antigen target of the antibody. Administered antibody contacts tumor cells, in other words, is placed in direct physical association with tumor cells, where the antibody can bind its target and deliver cytotoxic therapy.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. Examples of cancers that may be treated by the methods of the present disclosure include, but are not limited to, cancers of the lung, such as squamous cell lung carcinoma cells, cancers of the liver, such as hepatocellular carcinoma, and cancers of the skin, such as melanoma. Indeed, methods of the invention may be used to reduce the tumor size or metastasis, or both, of a tumor derived from, for example, a cancer chosen from lung, liver, skin, breast, colorectal, gastro-esophageal carcinoma, ovarian, prostate, renal, bladder, thyroid, squamous cell carcinoma of the head and neck, pancreatic, B cell lymphoma, multiple myeloma, non-Hodgkin's lymphoma (NHL), acute myeloid leukemia (AML), acute lymphoblastic leukemia (ALL), Hodgkin's Disease, or non-Hodgkin's lymphoma (NHL).

In various methods of the invention, a therapeutically effective amount of an antibody is administered to a subject in an amount sufficient to inhibit growth, replication or metastasis of cancer cells, or to inhibit a sign or a symptom of the cancer. Suitable subjects may include those diagnosed with a cancer, in which the tumor cells express a target entigen of an antibody according to the invention. A therapeutically effective amount of an antibody according to the invention will depend upon the severity of the cancer, and the general state of the patient's health. A therapeutically effective amount of the antibody is that which provides either subjective relief of a symptom(s) or an objectively identifiable improvement as noted by a clinician or other qualified professional.

Antibodies according to the invention, that are administered to subjects in need thereof, are formulated into compositions. More particularly, the antibodies can be formulated for systemic administration, or local administration, such as intra-tumor administration. For example, an antibody according to the invention may be formulated for parenteral administration, such as intravenous administration. The compositions can be prepared in unit dosage forms for administration to a subject. The amount and timing of administration are at the discretion of the treating clinician to achieve the desired outcome.

Administration of antibodies according to the invention can also be accompanied by administration of other anti-cancer agents, such as a chemotherapeutic agent, or therapeutic treatments, such as surgical resection of a tumor. Any suitable anti-cancer agent can be administered in combination with the antibodies disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and other antibodies that specifically target cancer cells.

The compositions for administration can include a solution of the antibody dissolved in a pharmaceutically acceptable carrier, such as an aqueous carrier. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, or glycerol as a vehicle. For solid compositions, such as powder, pill, tablet, or capsule forms, conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. The foregoing carrier solutions are sterile and generally free of undesirable matter, and may be sterilized by conventional, well known sterilization techniques. The compositions may contain pharmaceutically acceptable auxiliary substances as required to approximate physiological conditions such as pH adjusting and buffering agents, and toxicity-adjusting agents such as sodium acetate, sodium chloride, potassium chloride, calcium chloride, and sodium lactate. The concentration of antibody in these formulations can vary widely, and will be selected primarily based on fluid volumes, viscosities, patient body weight and the like in accordance with the particular mode of administration selected and the subject's needs.

Options for administering an antibody according to the invention include, but are not limited to, administration by slow infusion, or administration via an intravenous push or bolus. Prior to being administered, an antibody composition according to the invention may be provided in lyophilized form, and rehydrated in a sterile solution to a desired concentration before administration. The antibody solution may then be added to an infusion bag containing 0.9% sodium chloride, USP, and in some cases administered at a dosage of from 0.5 to 15 mg/kg of body weight. In one example of administration of an antibody composition according to the invention, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

Antibody compositions according to the invention may also be controlled release formulations. Controlled release parenteral formulations, for example, can be made as implants, or oily injections. Particulate systems, including microspheres, microparticles, microcapsules, nanocapsules, nanospheres, and nanoparticles, may also be used to deliver antibody compositions according to the invention. Microcapsules, as referred to herein, contain an antibody according to the invention as a central core component. In microspheres, an antibody according to the invention is dispersed throughout the particle. Particles, microspheres, and microcapsules smaller than about 1 μm are generally referred to as nanoparticles, nanospheres, and nanocapsules, respectively.

As described above, antibodies according to the invention may also be useful for diagnosing or monitoring the presence of a pathologic condition, such as, but not limited to, a cancer. More particularly, methods of the invention are useful for detecting expression the antigen target of an antibody according to the invention. Detection may be in vitro or in vivo. Any tissue sample may be used for in vitro diagnostic detection, including, but not limited to, tissue from biopsies, autopsies and pathology specimens. Biological samples include sections of tissues, for example, frozen sections taken for histological purposes. Biological samples further include body fluids, such as blood, serum, plasma, sputum, spinal fluid or urine.

A method determines if a subject has cancer by contacting a sample, such as a biopsy, from the subject with an antibody according to the invention; and detecting binding of the antibody to its target antigen present in the sample. An increase in binding of the antibody to its target antigen in the sample, as compared to binding of the antibody in a control sample identifies the subject as having cancer, for example, lung cancer, such as, squamous cell carcinoma of the lung, or hepatocellular carcinoma, or melanoma, or any other type of cancer, including the various cancers disclosed, above, that expresses the EPN1 antigen target of an antibody according to the invention. In general, a control sample is a sample from a subject without cancer.

Diagnostic methods differ in their sensitivity and specificity. The “sensitivity” of a diagnostic assay is the percentage of diseased individuals who test positive (percent of true positives). The “specificity” of a diagnostic assay is one minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (e.g., severity) of a pathologic condition, such as liver cancer or metastasis.

Antibodies of the invention can be linked to a detectable label to form immunoconjugates that are useful as diagnostic agents. A detectable label, as referred to herein, is a compound or composition that is conjugated directly or indirectly to an antibody according to the invention, for the purpose of facilitating detection of a molecule that correlates to presence of a disease, such as, for example, a tumor cell antigen that is the antigen target of an antibody according to the invention. Detectable labels useful for such purposes are well known in the art, and include: radioactive isotopes, such as ³⁵S, ¹¹C, ¹³N, ¹⁵O, ¹⁸F, ¹⁹F, technetium-99m (“^{99m}Tc), ¹³¹I, ³H, ¹⁴C, ¹⁵N, ⁹⁰Y, ¹¹¹In and ¹²⁵I; fluorophores; chemiluminescent agents; enzymatic labels, such as horseradish peroxidase, beta-galactosidase, luciferase, alkaline phosphatase; biotinyl groups; predetermined polypeptide epitopes recognized by a secondary reporter, such as a leucine zipper pair sequences, binding sites for secondary antibodies, metal binding domains, epitope tags; and magnetic agents, such as gadolinium chelates. A labeled antibody according to the invention may also be referred to as a “labeled antibody”. For some antibodies according to the invention, labels are attached by spacer arms of various lengths to reduce potential steric hindrance.

A diagnostic method comprising a step of using an antibody according to the invention may, in certain applications, be an immunoassay. While the details of immunoassays may vary with the particular format employed, a method of detecting the antigen target of an antibody according to the invention in a biological sample generally includes the steps of contacting the biological sample with the antibody which specifically reacts with the antigen, under immunologically reactive conditions to form an immune complex. The presence of the resulting immune complex can be detected directly or indirectly. In other words, an antibody according to the invention can function as a primary antibody (1° Ab) in a diagnostic method, and a labeled antibody, specific for the antibody according to the invention, functions as the 2° Ab. In the case of indirect detection of an immune complex, the use of an antibody according to the invention for a diagnostic method will also include the use of a labelled secondary antibody (2° Ab) to detect binding of the primary antibody—the antibody according to the invention—to its target antigen. Suitable detectable labels for a secondary antibody include the labels, described above, for directly labeled antibodies according to the invention. A 2° Ab, used in a diagnostic method according to the invention, may also be a “detector antibody”, as defined, above, for use in conjunction with an antibody according to the invention that contains a CH3 epitope tag, as described in PCT Patent Application No. PCT/US19/32780.

Antibodies according to the invention can also be used for fluorescence activated cell sorting (FACS). A FACS analysis of a cell population employs a plurality of color channels, low angle and obtuse light-scattering detection channels, and impedance channels, among other more sophisticated levels of detection, to separate or sort cells (see U.S. Pat. No. 5,061,620).

Reagents used in a diagnostic application of an antibody according to the invention, as described above, may be provided in a kit for detecting the antigen target of an antibody according to the invention a biological sample, such as a blood sample or tissue sample. Such a kit can be used to confirm a cancer diagnosis in a subject. For example, a diagnostic kit comprising an antibody according to the invention can be used to perform a histological examination for tumor cells in a tissue sample obtained from a biopsy. In a more particular example, a kit may include antibodies according to the invention that can be used to detect lung cancer cells in tissue or cells obtained by performing a lung biopsy. In an alternative, particular example, a kit may include antibodies according to the invention that can be used to detect hepatocellular carcinoma cells in a liver biopsy. In addition, an alternative, particular example, a kit may include antibodies according to the invention that can be used to detect melanoma in tissue or cells obtained by performing a biopsy.

Kits for detecting an antigen target of an antibody according to the invention will typically comprise an antibody according to the invention in the form of a monoclonal antibody, or a fragment thereof, such as an scFv fragment, a VH domain, or a Fab. The antibody may be unlabeled of labeled by a detectable marker, such as a fluorescent, radioactive, or an enzymatic label, as described above. A kit also generally includes instructional materials disclosing means of use of an antibody according to the invention. The instructional materials may be written, in an electronic form, such as a portable hard drive, and the materials also be visual, such as video files. Instructional materials may also refer to a website or link to an application software program, such as a mobile device or computer “app”, that provides instructions. A kit may also include additional components to facilitate the particular application for which the kit is designed. For example, a kit may also contain a means of detecting a label (such as enzyme substrates for enzymatic labels, filter sets to detect fluorescent labels, appropriate secondary labels such as a secondary antibody, or the like). Buffers and other reagents, which are routinely in methods of using an antibody according to the invention for diagnostic purposes, can also be included in a kit according to the invention.

Antibodies according to the invention can be produced by various recombinant expression systems. In other words, the antibodies can be produced by the expression of nucleic acid sequences encoding their amino acid sequences in living cells in culture. An “isolated” antibody according to the invention is one which has been substantially separated or purified away from other biological components environment, such as a cell, proteins and organelles. For example, an antibody may be isolated if it is purified to: i) greater than 95%, 96%, 97%, 98%, or 99% by weight of protein as determined by the Lowry method, and alternatively, more than 99% by weight; ii) a degree sufficient to obtain at least 15 residues of N-terminal or internal amino acid sequence by use of a spinning cup sequenator; iii) homogeneity by SDS-PAGE, under reducing or nonreducing conditions, using Coomassie blue or silver stain. Isolated antibody may also be an antibody according to the invention that is in situ within recombinant cells, since at least one component of the antibody's natural environment will not be present. Ordinarily, however, isolated antibody will be prepared by at least one purification step.

A variety of host-expression vector systems may be utilized to express an antibody according to the invention, by transforming or transfecting the cells with an appropriate nucleotide coding sequences for an antibody according to the invention. Examples of host-expression cells include, but are not limited to: Bacteria, such as E. coli and B. Subtilis, which may be transfected with antibody coding sequences contained within recombinant bacteriophage DNA, plasmid DNA, or cosmid DNA expression vectors; Yeast, such as Saccharomyces and Pichia, transformed with recombinant yeast expression vectors containing antibody coding sequences; Insect cell systems, infected with recombinant virns expression vectors, such as baculovinls, containing antibody coding sequences; Plant cell systems infected with recombinant vims expression vectors, such as cauliflower mosaic virus (“CaMV”), or tobacco mosaic vims (“TMV”), containing antibody coding sequences; and Mammalian cell systems, such as, but not limited to COS, Chinese hamster ovary (“CHO”) cells, ExpiCHO, baby hamster kidney (“BHK”) cells, HEK293, Expi293, 3T3, NSO cells, harboring recombinant expression constructs containing promoters derived from the genome of mammalian cell, such as the metallothionein promoter or elongation factor I alpha promoter, or from mammalian viruses, such as the adenovirus late promoter, and the vaccinia virus 7.5K promoter. For example, mammalian cells such as Human Embryonic Kidney 293 (HEK293) or a derivative thereof, such as Expi293, in conjunction with a dual promoter vector that incorporates mouse and rat elongation factor 1 alpha promoters to express the heavy and light chain fragments, respectively, is an effective expression system for antibodies according to the invention, which can be advantageously selected, depending upon the use intended for the antibody molecule being expressed.

When a large quantity of an antibody according to the invention is to be produced for the generation of a pharmaceutical composition of the antibody, vectors which direct the expression of high levels of readily purified fusion protein products may be desirable. Such vectors include, but are not limited to: a pUR278 vector (Ruther et al. EMBO J. 2:1791 (1983)), in which the antibody coding sequence may be ligated individually into the vector in frame with a lac Z coding region so that a fusion protein is produced; a pIN vector (Inouye & Inouye, Nucleic Acids Res. 13:3101-3109 (1985), and Van Heeke & Schuster, J. Biol. Chem. 24:5503-5509 (1989)); a pGEX vectors to fuse antibodies of the invention with glutathione S-transferase (“GST”). A GST fusion protein of an antibody according to the invention and a polypeptide tag is soluble and can easily be purified from lysed cells, by adsorption and binding to matrix glutathione-agarose beads, followed by elution in the presence of free glutathione. The pGEX vectors, by contrast, are designed to include thrombin or factor Xa protease cleavage sites so that the cloned target gene product—an antibody according to the invention—can be released from the GST moiety.

A host expression cell system may also be chosen which modulates the expression of inserted sequence(s) coding for an antibody according to the invention, or modifies and processes the gene product as desired. For example, modifications, including the glycosylation and processing, such as cleavage of protein products, may be important for the function of the protein. Indeed, different host cells have characteristic and specific mechanisms for the posttranslational processing and modification of proteins and gene products. To this end, eukaryotic host cells, which possess appropriate cellular machinery for proper processing of a primary transcript, as well as the glycosylation and phosphorylation of a gene product according to the invention may be used.

The vector used to produce an antibody according to the invention comprises a nucleic acid molecule encoding at least a portion of that particular antibody. For example, such a nucleic acid sequence can comprise a DNA sequence corresponding to SEQ ID NO: 3, or a portion thereof. Thus, a first nucleic acid encoding at least a portion of an antibody according to the invention, that is operably linked with a second nucleic acid sequence that is placed in a functional relationship with the first nucleic acid sequence, such as a promoter, is a nucleic acid according to the invention. An operable linkage exists if a linked promoter sequence affects the transcription or expression of the coding sequence. Generally, operably linked DNA sequences are contiguous and, may also join two, or more, protein-coding regions, in the same reading frame.

When a nucleic acid comprising a DNA sequence according to the invention is substantially separated or purified away from other biological components in the environment, such as a cell, other chromosomal and extra-chromosomal DNA and RNA, proteins and organelles, it may be considered to be an “isolated nucleic acid” according to the invention. For example, a nucleic acid, which has been purified by standard purification methods, is an isolated nucleic acid.

Nucleic acids according to the invention also include degenerate variants of nucleotides encoding an antibody according to the invention. More particularly, a “degenerate variant” refers to a polynucleotide, which encodes an antibody according to the invention, but is degenerate as a result of the genetic code. All degenerate nucleotide sequences are included, according to the invention, as long as the amino acid sequence of the encoded antibody specifically binds the antigen target of an antibody according to the invention.

### EXAMPLES

The following Examples describe the isolation and characterization of an antibody, IMM20059, that binds to EPN1.

### Example 1. Isolation of a Human Hybridoma Producing an Antibody that Binds the Surface of Intact Human Cancer Cells

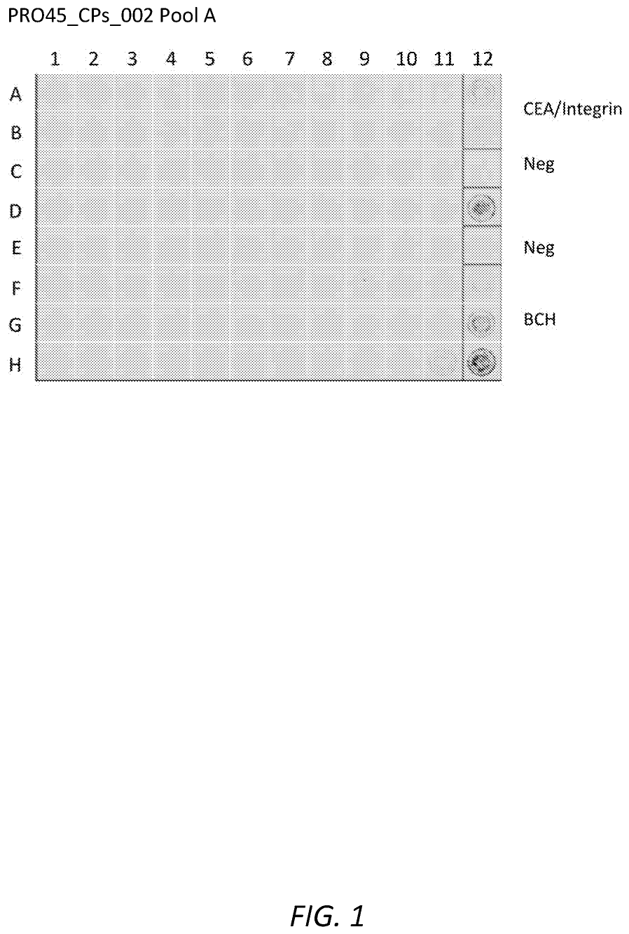

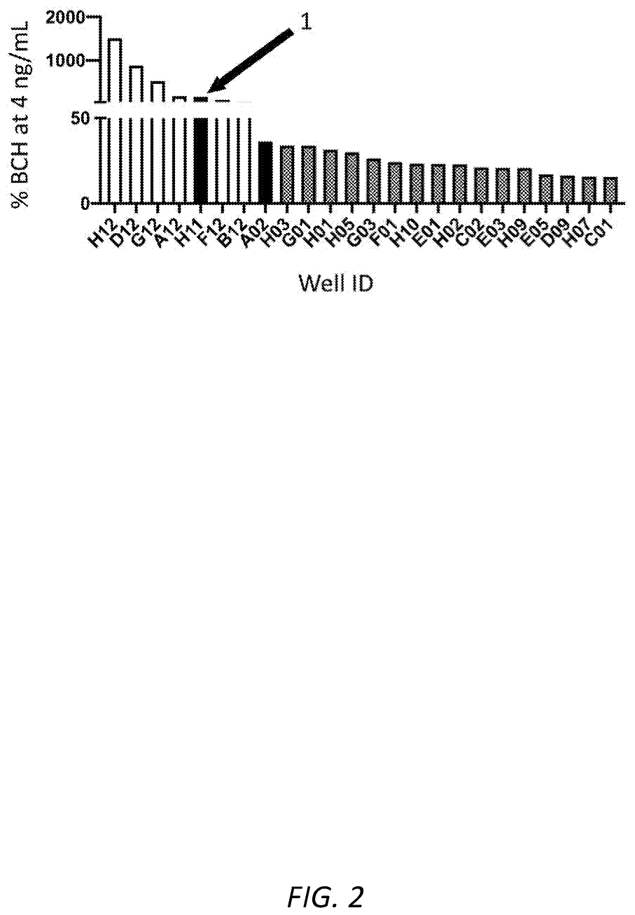

FIG. 1 shows the detection of Abs produced by PR045-2H11 hybridoma cells, which were generated from the fusion of human B cells isolated from the lymph node of a head and neck cancer patient, with a B5-6T fusion partner cell line. Fusion of human B cells with B5-6T cells was carried out by electrofusion essentially as described in U.S. Pat. No. 8,999,707 (“Method of making hybrid cells that express useful antibodies”). Post-fusion, hyridomas were plated and allowed to grow for approximately two weeks. Conditioned media from IgG-positive hybridomas were then collected and screened for the ability of the antibodies to bind to the surface of cancer cell lines. Binding of PR045-2H11-produced Abs to pools of live, intact cancer cell lines was detected using fluorophore-labelled goat anti-human IgG secondary Abs in combination with a LI-COR Odyssey™ Sa imaging system configured for 96-well plates. Prior to screening, the cancer cells were mixed in equal proportions and the pools were aliquoted into 96-well plates and allowed to attach for 24-48 hours. Hybridoma supernatants were diluted in media containing sodium azide at a final concentration of 0.1%. Three different positive controls were used to assess activity in the screening assay: 1) a mixture of anti-basigin, anti-EGFR, and anti-ERBB2 (BCH) in equal ratios was plated in a 3-point, 1:5 serial dilution, starting with a concentration of 100 ng/ml each; 2) a two-point titration of a mix of anti-CEA and anti-integrin mAbs containing either 100 ng/mL or 20 ng/mL of each antibody; and 3) a single concentration of an anti-CD29 mAb (500 ng/mL). Secondary antibody, alone, was used as a negative control. The combination of controls provided a range of absolute signal intensities across both the cell line pools and the detection range of the LI-COR instrument. Detection signals for PR045-2H11 exhibited a 177% increase in signal as compared to baseline positive control. All wells containing hybridomas that exhibited, at least, a 15% increase in signal as compared to baseline positive control are represented in FIG. 2.

### Example 2. The PR045-2H11 Hybridoma Produces an IgG with IGHV3/IGK Variable Domains

Nucleotide sequences, encoding the variable heavy chain (V_{H}) and variable light chain (V_{L}) domains of the PR045-2H11-produced Ab, were obtained by RT-PCR amplification of RNA isolated from PR045-2H11 hybridoma cells, and subjecting the resulting antibody cDNA to sequencing reactions. SEQ ID NO: 1 corresponds to the V_{H} and SEQ ID NO: 5 corresponds to the V_{L} of PR045-2H11-produced Ab. The PCR strategy used here was not designed to amplify regions corresponding to the 5′ most portion of framework 1 of the variable domains. Ig heavy chain V (“IGHV”) and Immunoglobulin kappa locus (“IGKL”) gene assignments were predicted based upon homology to known germline gene sequences, and used as surrogates for the bona fide 5′ ends of the V_{H} and V_{L} sequences.

The coding region of the V_{H} and V_{L} domains have the hallmarks of somatic hypermutation, differing from germline sequences by 15 and 14 nucleotides respectively. A full-length expression fragment for PR045-2H11 V_{H} (SEQ ID NO: 3) was generated using germline sequence corresponding to 5′ end of framework 1 of IGHV3-48*02. A full-length expression fragment for PR045-2H11 V_{L} (SEQ ID NO: 7) was generated using the germline sequence corresponding to the 5′ end of framework 1 of IGKV3-11*01. Fragments corresponding to SEQ ID NO: 3 and SEQ ID NO: 7 domains were synthesized with additional 5′ and 3′ extensions to facilitate Gibson-style cloning into a dual promoter IgG1 expression vector. The corresponding protein sequences encoded by the V_{H} and V_{L} fragments are defined in SEQ ID NO: 4 and SEQ ID NO: 8, respectively. Similarly, DNA fragments encoding the V_{H} (SEQ ID NO: 4) and V_{L} (SEQ ID NO: 8) of PR045-2H11 were cloned into a human IgG1 two vector expression system. The full-length amino acid sequence of the heavy and light chains encoded by both vector systems correspond to SEQ ID NO 15 and SEQ ID NO: 16, respectively

### Example 3: IMM20059, the Recombinant Form of the PR045-2H11 Ab, Binds the Surface of Tumor Cell Lines

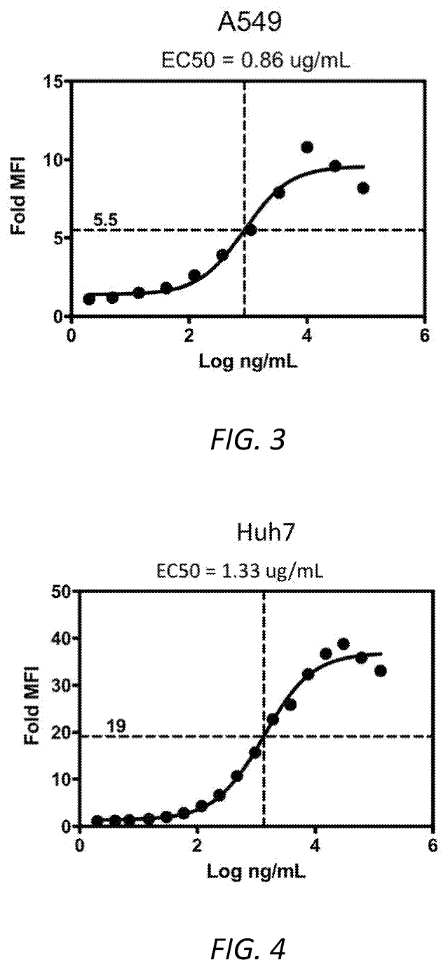

Full-length IgG1 antibody, containing the PR045-2H11 Ab V_{H} and V_{L} domains, was expressed recombinantly by transient transfection into mammalian cell lines, such as Chinese Hampster Ovary (CHO) and human embryonic kidney (HEK), or derivatives of those cell lines, using standard conditions. The recombinant antibody, referred to as IMM20059, was purified from conditioned media by affinity chromatography, buffer exchanged into PBS and analyzed for activity by flow cytometry. IMM20059 displayed binding activity consistent with the original PR045-2H11 hybridoma-produced antibody. IMM20059 binds, to varying degrees, to the surfaces of cells included in a panel of human cancer cells, immortalized normal human cell lines, and mouse tumor lines (Table 1). As depicted in FIGS. 3 and 4, IMM20059 displays saturable binding to the surface of A549 lung adenocarcinoma and Huh7 hepatocellular carcinoma cell lines, respectively, when analyzed by flow cytometry. IMM20059 binds to A549 and Huh7 with an EC50 of 0.9 and 1.3 μg/mL, respectively. These values correspond to EC50 values of between 6-9 nM.

TABLE 1
IMM20059 binding to various cell lines

| Human | | | Human | | | Mouse | | |
|---|---|---|---|---|---|---|---|---|
| Tumor Line | MFI | STD | Normal Line | MFI | STD | Tumor Line | MFI | STD |
| OE21 | 46 | 27 | MRC5 | 9 | N/A | 4T1 | 8 | 0 |
| HepG2 | 39 | 47 | CCD841con | 8 | N/A | EMT6 | 6 | 3 |
| H460 | 30 | 5 | HEK293 | 5 | N/A | CT-26 | 6 | 4 |
| Huh7 | 26 | 9 | BJ | 3 | N/A | B16F0 | 5 | 2 |
| P29 Clone1 | 21 | N/A | | | | | | |
| H1735 | 21 | 6 | | | | | | |
| 22Rv1 | 20 | 4 | | | | | | |
| WM1366 | 17 | 2 | | | | | | |
| MFE296 | 17 | 10 | | | | | | |
| OVCAR3 | 16 | N/A | | | | | | |
| H660 | 16 | 2 | | | | | | |
| SKMEL28 | 15 | 1 | | | | | | |
| 769p | 14 | 11 | | | | | | |
| A431 | 14 | 10 | | | | | | |
| SW480 | 13 | 6 | | | | | | |
| CAL27 | 13 | 13 | | | | | | |
| RT4 | 12 | N/A | | | | | | |
| DMS114 | 12 | 11 | | | | | | |
| LNCAP | 11 | 1 | | | | | | |
| ES2 | 9 | 6 | | | | | | |
| CAOV3 | 8 | N/A | | | | | | |
| A549 | 8 | 4 | | | | | | |
| TOV112D | 7 | 4 | | | | | | |
| 786o | 7 | N/A | | | | | | |
| ACHN | 7 | N/A | | | | | | |
| PC3 | 7 | 1 | | | | | | |
| FLO1 | 6 | 2 | | | | | | |
| HT29 | 6 | 6 | | | | | | |
| Calu6 | 6 | 3 | | | | | | |
| T47D | 5 | 2 | | | | | | |
| OE19 | 5 | 3 | | | | | | |
| NTERA | 5 | N/A | | | | | | |
| HCT116 | 5 | 2 | | | | | | |
| SCC15 | 5 | N/A | | | | | | |
| A375 | 5 | 3 | | | | | | |
| FADU | 5 | 3 | | | | | | |
| DU145 | 4 | 0 | | | | | | |
| MDAMB231 | 4 | N/A | | | | | | |
| N87 | 4 | N/A | | | | | | |
| WM3211 | 3 | N/A | | | | | | |
| HeLa | 3 | 1 | | | | | | |
| PSN1 | 3 | N/A | | | | | | |
| HT1197 | 3 | N/A | | | | | | |
| H522 | 3 | 2 | | | | | | |
| MCF7 | 2 | N/A | | | | | | |
| HCT15 | 2 | N/A | | | | | | |

MFI = Fold Mean Fluorescence Index above isotype control;
STD = Standard deviation;
N/A = not completed a sufficient number of times to generate STD

### Example 4. IMM20059 Binds to EPN1

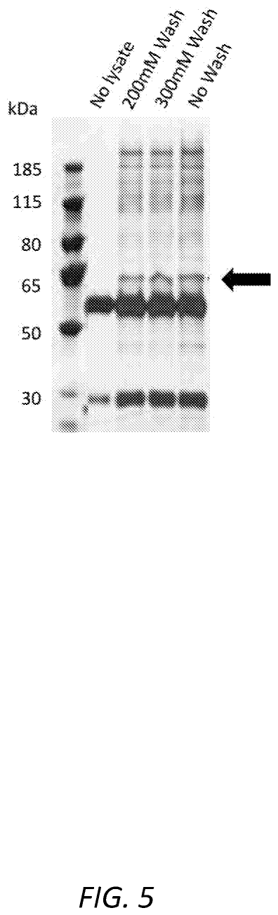

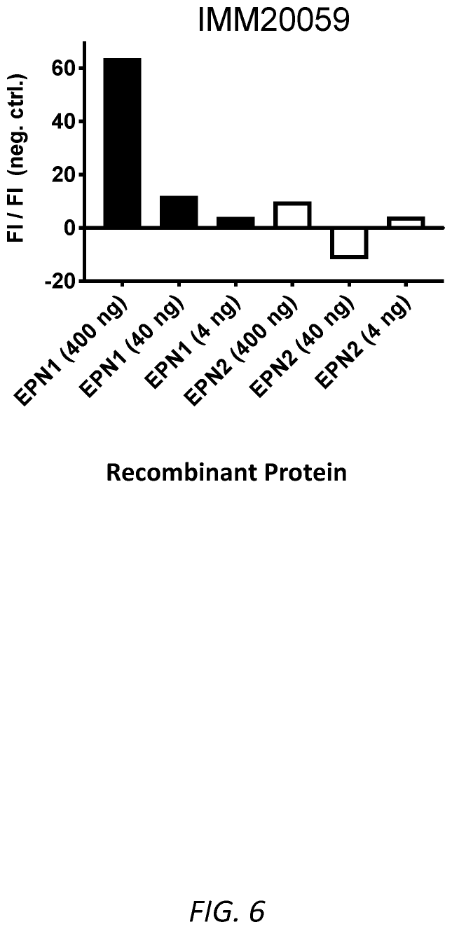

Immunoprecipitation experiments, carried out to identify the target antigen bound by IMM20059, consistently identified a band of approximately 65 kDa protein (FIG. 5). Mass-spectrometry analysis of the immunoprecipitated band identified the protein as EPN1. The ability of IMM20059 to bind EPN1 was confirmed in a series of in vitro assays. As depicted in FIG. 6, IMM20059 binds selectively, in a dose-dependent manner, to recombinant EPN1 as compared to its homolog EPN2.

IMM20059 was then screened using the CDI Human Proteome (HuProt) microarry-based High-Spec antibody cross-reactivity assay. In the assay, proteins corresponding to approximately 80% of the human proteome were spotted in native format onto microarrays and used to probe specificity of IMM20059. More specifically, IMM20059 (1 μg/mL) was incubated overnight at 4° C. against the native CDI HuProt array, with EPN1 (Origene, cat #TP307099) added to the array as an additional control. Slides were washed and IMM20059 binding was detected with an Alexa-647 conjugated anti-H+L secondary antibody. Non-specific hits that were bound by the secondary were eliminated from any analysis. Selective binding to target proteins were analyzed by a combination of overall signal intensity, Z-score to determine reproducibility of binding to replicates on each slide, and S-score to determine difference in selectivity versus possible targets. An S-score >3 between the top and second-ranked hits is considered as indicative of specificity for the top hit.

As shown in Table 2. IMM20059 bound to both EPN1 normally found on the array and the added control spot of EPN1, as two of the top six hits on the array. The signal intensity exceeded the maximal threshold for each of those six proteins, suggesting the potential for some cross-reactivity with alternate proteins. IMM20059 displayed selectivity for EPN1 as compared to EPN3 in the High-Spec assay. EPN3 is present on the protein array but did not exhibit selective binding.

TABLE 2
IMM20065 binding to human proteome in protein microarray format
CDI Native Array

| Rank | Protein | Z Score | S Score | F635 |
|---|---|---|---|---|
| 1 | EPN1 | 50.022 | 0 | 65535 |
| 2 | EPN1 | 50.022 | 0 | 65535 |
| 3 | SF3B4 | 50.022 | 0 | 65535 |
| 4 | RALGDS | 50.022 | 0 | 65535 |
| 5 | MAPK7 | 50.022 | 0 | 65535 |
| 6 | C4orf19 | 50.022 | 12.593 | 65535 |
| 7 | WIPF1 | 37.429 | 9.67 | 49060 |
| 8 | SH3BP1 | 27.759 | 6.119 | 36409 |
| 9 | RBM12 | 21.64 | 2.134 | 28404 |
| 10 | DBN1 | 19.506 | 0.708 | 25611 |

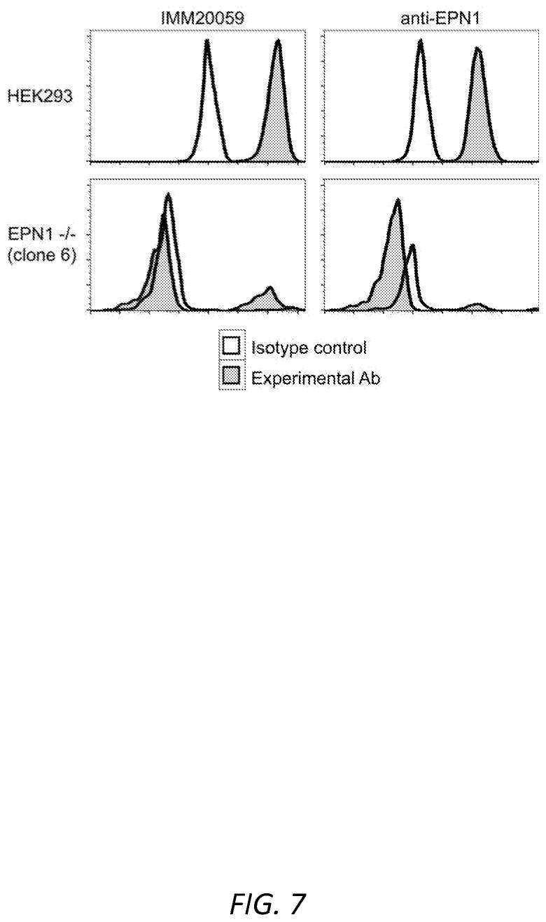

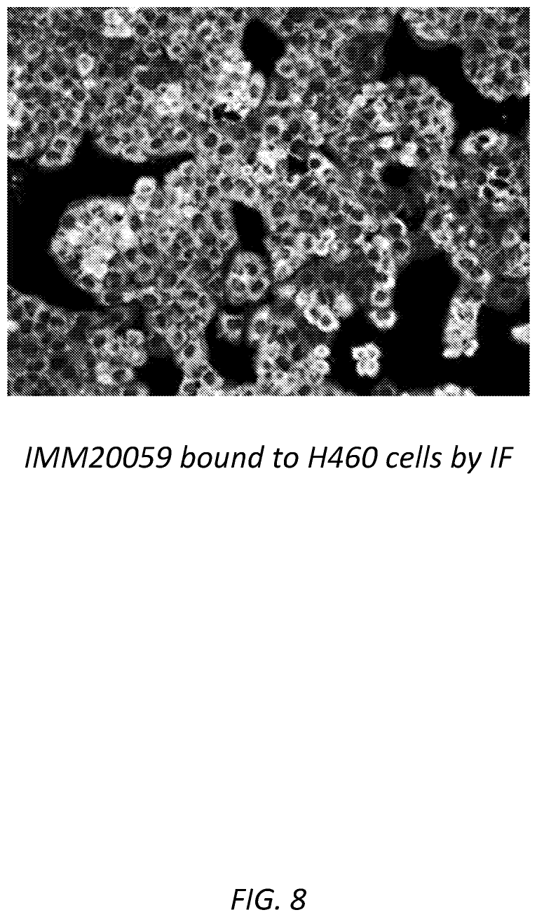

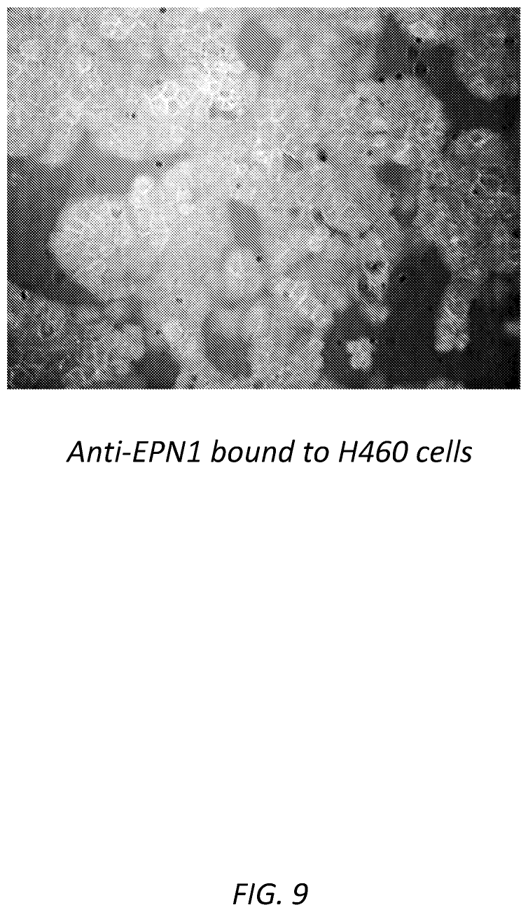

CRISPR-based disruption of the EPN1 gene in HEK293 cells aborgates the capacity of IMM20059 to bind to those cells. Flow cytometry-based analysis of fixed and permeabilized parental and EPN1−/− cells was carried out with IMM20059 and a commercially available anti-EPN1 antibody. Both antibodies demonstrate equivalent levels of binding to parental HEK293 cells that is lost in the EPN1−/− clone (FIG. 7). Residual binding to the EPN1−/− clone by both the commercial anti-EPN1 mAb and IMM20059 suggests that either the clone is actually a mixed population that includes EPN1-expressing cells or that the two antibodies cross-react, to a similar extent, with a non-EPN1 target protein.

IMM20059 and a commercially available mouse anti-huEPN1 were used as primary antibodies to visualize the localization of EPN1 in the H460 human lung cancer cell line. Consistent with the known membrane localization of EPN1, both antibodies showed membrane staining when visualized by immunofluoresnce using fluorophore-labelled secondary antibodies with appropriate species specificity to detect bound primary antibodies (FIGS. 7 & 8).

The strength of the interaction with recombinant EPN1 was further defined by surface plasmon resonance on a BIAcore2000 at 25° C. in standard running buffer (10 mM HEPES, 150 mM NaCl, 0.005% Tween-20, 0.2 mg/mL BSA, pH 7.4). Protein A surfaces were regenerated between binding cycles with 150 mM phosphoric acid. IMM20059, or an isotype control, were captured on CM5/Protein A sensor surface to generate binding and control surfaces; IMM20059 was captured at approximately 200 and 450 RU, the isotype control was captured to a density of approximately 600 RU. Binding of recombinant EPN1 to each of the three surfaces was tested, in triplicate, using a three-fold dilution series starting with 33.3 nM as the highest concentration. Binding of EPN1 was observed to both the high (450 RU) and low (200 RU) density IMM20059 surfaces. No binding was observed to the isotype control surface, under these conditions, at any concentration tested. Double-subtracted data obtained from the binding measured against the 450 RU IMM20059 surface was fit to a 1:1 binding model. As outlined in Table 3, IMM20059 demonstrated reproducible binding to EPN1 with an average K_{D} of 950+/−10 pM.

TABLE 3
Binding parameters determined for IMM20059/EPN1 at 25° C.

| Test | kₐ (M⁻¹s⁻¹) | k_{d} (s⁻¹) | K_{D} (pM) |
|---|---|---|---|
| 1^{st} | 7.3(2)e5 | 7.05(7)e−1 | 960(20) |
| 2^{nd} | 7.87(7)e5 | 7.36(7)e−4 | 940(10) |
| 3^{rd} | 7.6(1)e5 | 7.21(8)e−4 | 950(10) |
| Average | 7.6[3]e5 | 7.2[2]e−4 | 950[10] |

The numbers in parentheses are the errors in the last digits for the fits determined in the individual tests. The numbers in brackets are the experimental errors determined across the three tests.

Consistent with dot blot binding data, SPR analysis demonstrated that IMM20059 selectively binds to EPN1 as compared to EPN2. IMM20059 was captured at four different surface densities (500, 1000, 2600, and 2800 RU) on a CM5/Protein A sensor chip using 10 mM HEPES, pH 7.4, 150 mM NaCl, 0.005% Tween-20, 0.2 mg/mL BSA as a running buffer. EPN1 (Origene Cat #TP307099) or EPN2 (Origene Cat #TP310899) were diluted in running buffer to both 200 nM and 20 nM and analyzed for the ability to bind to the immobilized IMM20059 at 25° C. Under all of these conditions, IMM20059 bound strongly to EPN1 (Table 3) but failed to show any binding to EPN2. However, as detailed in Table 4, the observed off-rates for binding to EPN1 were dependent upon IMM20059 surface density, with the off-rate on the 500 RU surface being 2.4-times faster than the off-rate measured on the 2800 RU density surface. These data suggest the possibility that EPN1 may be multimerizing, either in solution or upon binding to the chip surface, which could induce an avidity effect to the binding interaction and alter the apparent K_{D} of the interaction.

TABLE 4
Binding of EPN1 to various surface densities of IMM20059

| Density (RU) | kₐ (M⁻¹s⁻¹) | k_{d} (s⁻¹) | K_{D} (pM) |
|---|---|---|---|
| 2800 | 2.93(2)e5 | 2.43(5)e−4 | 827(2) |
| 2600 | 2.54(3)e5 | 2.67(8)e−4 | 1050(3) |
| 1000 | 2.98(3)e5 | 4.54(8)e−4 | 1521(3) |
| 500 | 2.44(4)e5 | 5.87(1)e−4 | 2404(5) |

The numbers in parentheses are the errors in the last digits for the fits determined in the individual tests.

Binding of EPN1 to IMM20059 was evaluated when antibody was captured, in standard running buffer, onto the surface of a C1/Protein A sensor chip at a density of approximately 50 RU. The combination of the carboxymethylated, matrix-free surface of the C1 chip and the low density of IMM20059 is predicted to limit avid binding. A three-fold dilution series of EPN1 up to 200 nM in running buffer was passed over the surface at 25° C. and shown to bind to IMM20059 surface with a comparable on-rate (Table 5). However, the observed off-rate was approximately an order of magnitude faster under these conditions, leading to an approximately 10-fold decrease in the measured intrinsic binding affinity.

TABLE 5
Binding parameters determined for IMM20059/EPN1
at 25° C. on C1/Protein A chip

| Antigen | kₐ (M⁻¹s⁻¹) | k_{d} (s⁻¹) | K_{D} (nM) |
|---|---|---|---|
| EPN1 | 2.99(8)e5 | 3.03(1)e−3 | 10.1(3) |

The numbers in parentheses are the errors in the last digits for the fits determined in the individual tests.

### Example 5. IMM20059 Binds to Residues within a Highly Conserved Region of the EPN1

Cross-linking experiments were carried out to determine, with high resolution, the epitope on EPN1 that is bound by IMM20059. A GST-EPN1 fusion protein (Novus Biologicals, Cat #H00029924-P01; SEQ ID NO:17) served as the target antigen for the cross-linking experiments. The IMM20059/EPN1 protein complex was formed, incubated with deuterated cross-linkers, and subjected to multi-enzymatic cleavage with Trypsin, Chymotrypsin, ASP-N, Elastase and Thermolysin. After enrichment of the cross-linked peptides, the samples were analyzed by high resolution mass spectrometry (nLC-LTQ-Orbitrap MS) and the data generated were analyzed using XQuest and Stavrox software.

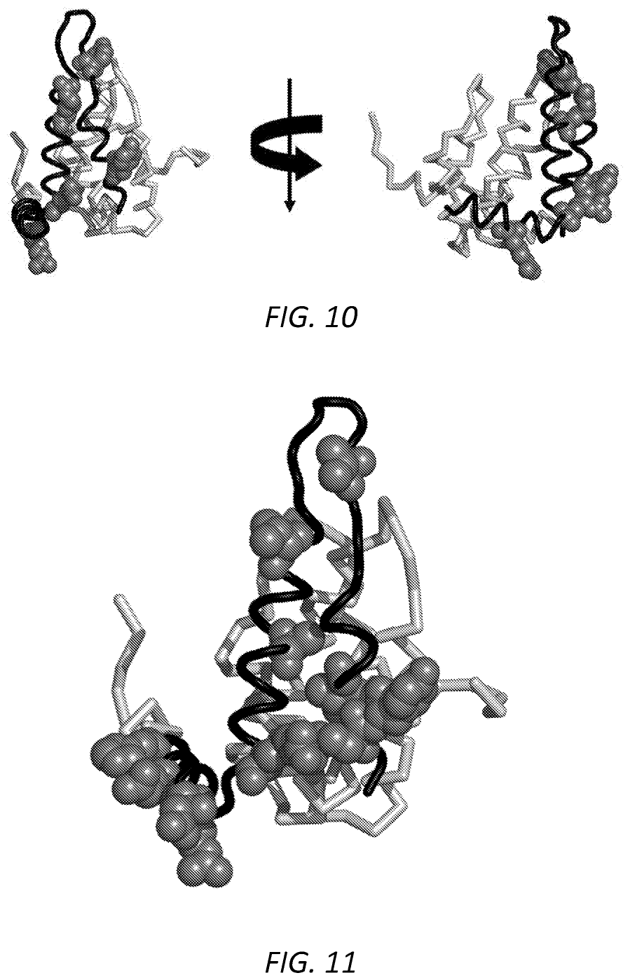

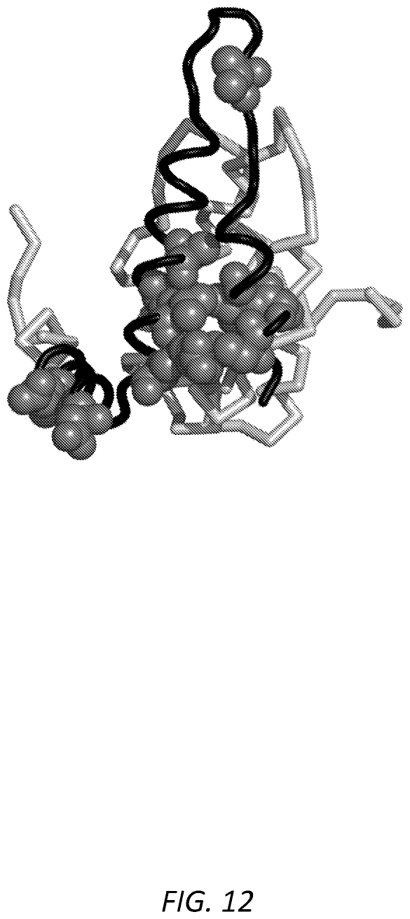

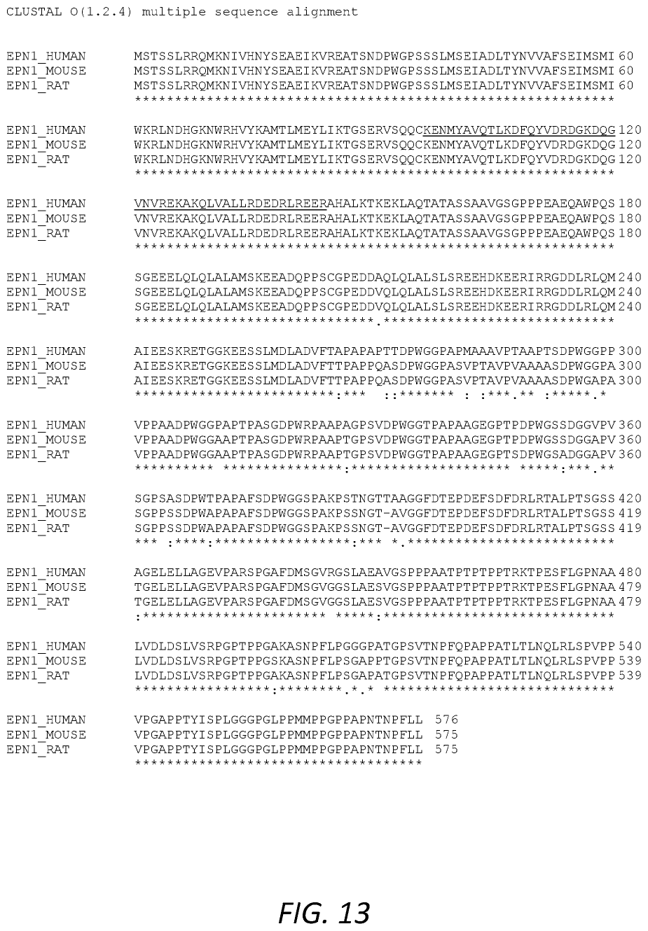

The nLC-orbitrap MS/MS analysis detected eight cross-linked peptides between EPN1 and IMM20059 (Table 6). The interactions map to two regions of the Epsin N-terminal Homology (“ENTH”) domain of EPN1, with cross-links being identified at amino acid positions 101, 111, 124, 135, and 141 of SEQ ID NO:18. The physical location of those cross-links within the ENTH domain were modeled onto the crystal structure of the rat EPN1 ENTH domain (RCSB PDB: 1EDU). Human EPN1 is 100% identical across the ENTH domain, and greater than 96% identical overall, to both rat and mouse EPN1 (FIG. 13). As depicted in FIG. 10, the residues identified as cross-linking to EPN1 sit on a single face of the ENTH domain in physical proximity to each other.

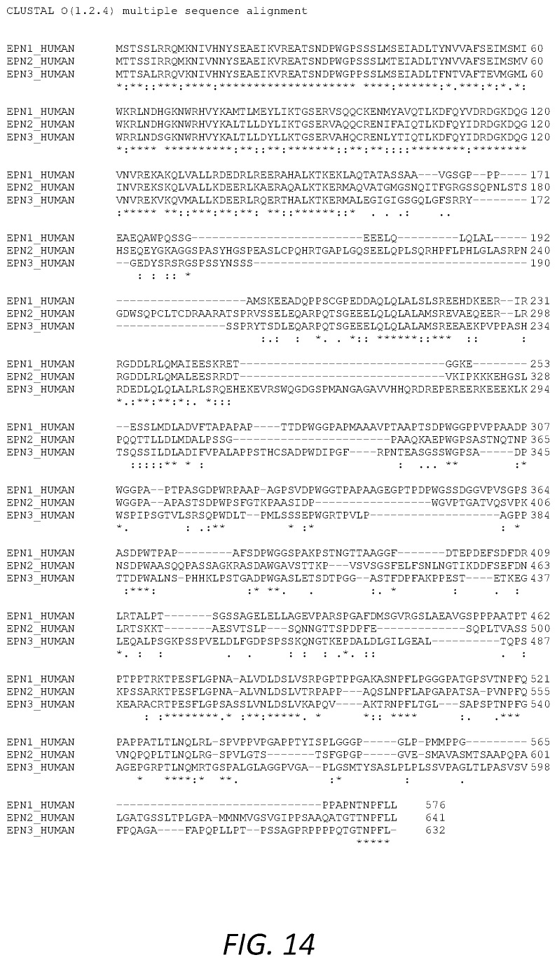

In contrast to the identity seen in EPN1 between species, human EPN1 is only 56.7% and 49.6% identical to human EPN2 and EPN3, respectively (FIG. 14). As depicted in FIGS. 11 & 12, a number of the amino acid differences map to the interface identified as binding to IMM20059. These differences provide a rationale for the selectivity of IMM20059 for EPN1 as compared to either EPN2 or EPN3.

TABLE 6
Crosslinked peptides identified between IMM20059 and EPN1

| | | IMM20059 peptides | | | |
|---|---|---|---|---|---|
| | Cleavage | Amino | Variable | | EPN1 |
| Sequences of linked peptides | Enzyme | acids | domain | CDR | peptides |
| GLEWVSYISSNSTTIYYADSVKGRFTISR | Trypsin | 44-72 | HC | H2 | 98-107 |
| (SEQ ID NO: 19)- | | | | | |
| ENMYAVQTLK (SEQ ID NO: 26) | | | | | |
| YNWLTFGGGTK (SEQ ID NO: 20)- | Trypsin | 92-102 | LC | L3 | 108-114 |
| DFQYVDR (SEQ ID NO: 27) | | | | | |
| GLEWVSYISSNSTTIYYADSVKGR | Trypsin | 44-67 | HC | H2 | 108-117 |
| (SEQ ID NO: 21)- | | | | | |
| DFQYVDRDGK (SEQ ID NO: 28) | | | | | |
| ATGIPAR (SEQ ID NO: 22)- | Trypsin | 55-61 | LC | L2 | 115-126 |
| DGKDQGVNVREK (SEQ ID NO: 29) | | | | | |
| LSCAASGFTFSIHSLNWVR | Trypsin | 20-38 | HC | H1 | 118-126 |
| (SEQ ID NO: 23)-DQGVNVREK | | | | | |
| (SEQ ID NO: 30) | | | | | |
| ATLSCR (SEQ ID NO: 24)- | Trypsin | 19-24 | LC | L1 | 127-139 |
| AKQLVALLRDEDR (SEQ ID NO: 31) | | | | | |
| SIHSLNW (SEQ ID NO: 25)- | Chymo- | 30-36 | HC | H1 | 135-154 |
| RDEDRLREERAHALKTKEKL | trypsin | | | | |
| (SEQ ID NO: 32) | | | | | |

Underlined peptide sequences in each complex correspond to the IMM20059-derived sequence.
The peptides corresponding with SEQ ID NOS: 21 and 28 were identified via two seperate crosslinked species.
Amino acid numbering is relative to SEQ ID NOs 16, 18, and 20

The amino acid identity between mouse and human EPN1, within the IMM20059 binding site, predicts that IMM20059 can bind mouse EPN1. This prediction is consistent with flow cytometry-based binding observed against mouse cancer cell lines (Table 1). FIG. 15 demonstrates that the surface pool of EPN1, on both the human cell line MFE296 and the mouse cell line NIH/3T3, represents a fraction of the total cellular EPN1. Cells were either stained as live cells, to identify surface EPN1, or permeabilized to identify both surface and intracellular pools. IMM20059 and a commercially available anti-EPN1 monoclonal antibody (clone C-11) exhibited similar staining patterns.

### Example 6. Loss of EPN1 Activity Inhibits Cell Growth

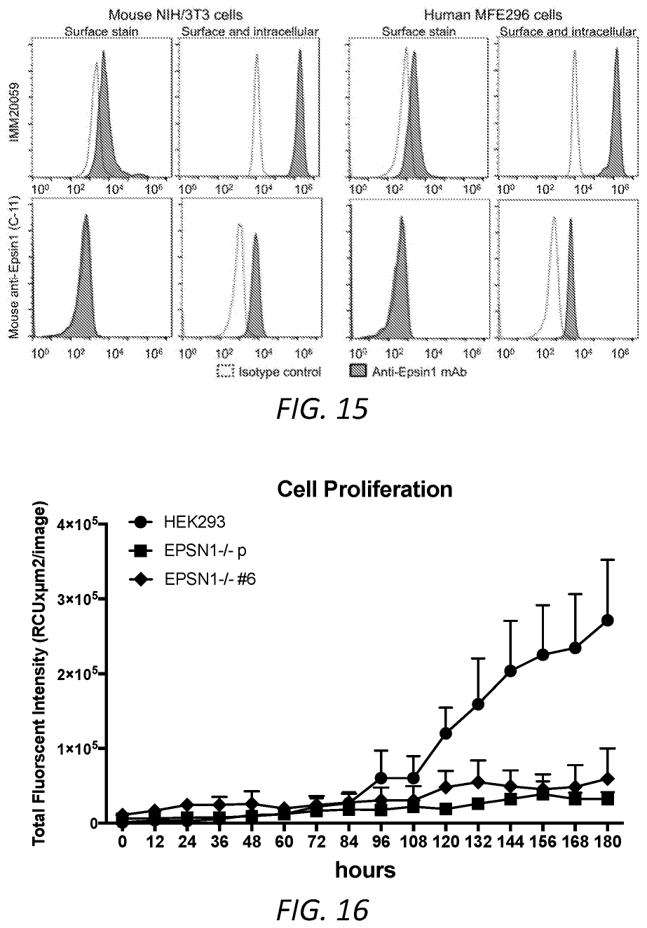

Multiple clones, harboring CRISPR-based knock-outs of the EPN1 gene, were analyzed in cell proliferation assays. As depicted in FIG. 16, all clones exhibited a statistically significant decrease in cell growth rate as compared to the parental HEK293 cells. This supports the hypothesis that disruption of EPN1 function, potentially with an antibody-based approach, could slow the growth of cancer cells.

### Example 7. IMM20059 Slows the Tumor Growth in a Syngeneic Model of Melanoma

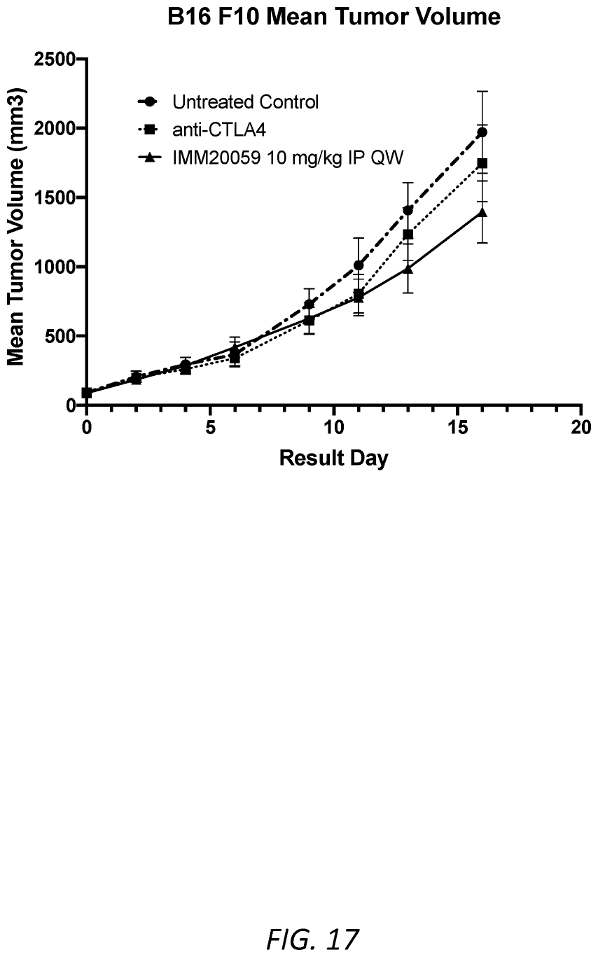

The B16F0 melanoma model, grown as syngeneic tumors in C57Bl/6 mice, were used to evaluate the impact of IMM20059 on tumor growth. Mice harboring established B16F10 tumors (n≥8/cohort) were treated via weekly intraperitoneal injection of IMM20059 at a dose of 10 mg/kg and mean tumor volumes calculated via caliper measurements. As depicted in FIG. 17, the growth of IMM20059-treated tumors was significantly slowed as compared to the growth of vehicle-treated tumors. An antibody targeting CTLA4, a clinically relevant immuno-oncology checkpoint, which is known to modestly inhibit the growth of B16F10 tumors, was used as a positive control in this experiment.

Sequence Listing
V_{H} PR045-2H11 nucleotide sequence
SEQ ID NO: 1
GACTCTCCTGTGCAGCCTCTGGATTCACCTTCAGTATCCATAGCCTGAATT
GGGTCCGCCAGGCTCCAGGGAAGGGACTGGAGTGGGTTTCGTATATTAGTA
GTAACAGTACTACCATATATTACGCAGACTCTGTGAAGGGCCGATTCACCA
TCTCCAGAGACAATGCCAAGGACTCCCTGTATCTGCAAATGAACAGCCTCA
GAGACGAGGACACGGCTGTATATTACTGTGCGAGAGACTACTACTGTACTG
GTGGTACCTGCTTCTTTCTTCCTGACCTCTGGGGCCGGGGAGCCCTGGTCA
CCGTCTCCTCAGCCTCCACCAAGGGCCCATCGGTCTTCCCCCTGGCGC
V_{H} PR045-2H11 amino acid sequence
SEQ ID NO: 2
LSCAASGFTFSIHSLNWVRQAPGKGLEWVSYISSNSTTIYYADSVKGRFTI
SRDNAKDSLYLQMNSLRDEDTAVYYCARDYYCTGGTCFFLPDLWGRGALVT
VSSASTKKGPSVFPLA
V_{H} PR045-2H11 expression fragment nucleotide
sequence
SEQ ID NO: 3
ACAGGCGCGCACTCCGAGGTGCAGCTGGTGGAGTCTGGGGGAGGCTTGGTA
CAGCCTGGGGGGTCCCTGAGACTCTCCTGTGCAGCCTCTGGATTCACCTTC
AGTATCCATAGCCTGAATTGGGTCCGCCAGGCTCCAGGGAAGGGACTGGAG
TGGGTTTCGTATATTAGTAGTAACAGTACTACCATATATTACGCAGACTCT
GTGAAGGGCCGATTCACCATCTCCAGAGACAATGCCAAGGACTCCCTGTAT
CTGCAAATGAACAGCCTCAGAGACGAGGACACGGCTGTATATTACTGTGCG
AGAGACTACTACTGTACTGGTGGTACCTGCTTCTTTCTTCCTGACCTCTGG
GGCCGGGGAGCCCTGGTCACCGTCTCCTCAGCCTCCACCAAGGGCCCATC
V_{H} PR045-2H11 expression fragment amino acid
sequence
SEQ ID NO: 4
EVQLVESGGGLVQPGGSLRLSCAASGFTFSIHSLNWVRQAPGKGLEWVSYI
SSNSTTIYYADSVKGRFTISRDNAKDSLYLQMNSLRDEDTAVYYCARDYYC
TGGTCFFLPDLWGRGALVTVSSASTKGPSVFPL
V_{L} PR045-2H11 nucleotide sequence
SEQ ID NO: 5
AAGAGCCACCCTCTCCTGCAGGGCCAGTCAGAATATCAGCAACTTCTTAGC
CTGGTACCAACACAAACCTGGCCAGGCTCCCAGGCTCCTCATCTATGATGC
ATCCATCAGGGCCACTGGCATCCCAGCCAGGTTCAGTGGCAGTGGGTCTGG
GACAGACTTCAGTCTCACCATCAGCAGCCTGGAGCCTGAAGATTTTGCAGT
TTATTTCTGTCAGCAGCGTTACAACTGGCTCACTTTCGGCGGAGGGACCAA
GGTAGAGATCAAACGAACTGTGGCTGCACCATCTGTCTTCATCT
V_{L} PR045-2H11 amino acid sequence
SEQ ID NO: 6
RATLSCRASQNISNFLAWYQHKPGQAPRLLIYDASIRATGIPARFSGSGSG
TDFSLTISSLEPEDFAVYFCQQRYNWLTFGGGTKVEIKRTVAAPSVFI
V_{L} PR045-2H11 expression fragment nucleotide
sequence
SEQ ID NO: 7
TCAGATACCTCCGGAGAAATTGTGTTGACACAGTCTCCAGCCACCCTGTCT
TTGTCTCCAGGGGAAAGAGCCACCCTCTCCTGCAGGGCCAGTCAGAATATC
AGCAACTTCTTAGCCTGGTACCAACACAAACCTGGCCAGGCTCCCAGGCTC
CTCATCTATGATGCATCCATCAGGGCCACTGGCATCCCAGCCAGGTTCAGT
GGCAGTGGGTCTGGGACAGACTTCAGTCTCACCATCAGCAGCCTGGAGCCT
GAAGATTTTGCAGTTTATTTCTGTCAGCAGCGTTACAACTGGCTCACTTTC
GGCGGAGGGACCAAGGTAGAGATCAAACGAACTGTGGCTG
V_{L} PR045-2H11 expression fragment amino acid
sequence
SEQ ID NO: 8
EIVLTQSPATLSLSPGERATLSCRASQNISNFLAWYQHKPGQAPRLLIYDA
SIRATGIPARFSGSGSGTDFSLTISSLEPEDFAVYFCQQRYNWLTFGGGTK
VEIKRTVA
H-CDR1
SEQ ID NO: 9
SIHSLN
H-CDR2
SEQ ID NO: 10
YISSNSTTIYYADSVKG
H-CDR3
SEQ ID NO: 11
DYYCTGGTCFFLPDL
L-CDR1
SEQ ID NO: 12
RASQNISNFLA
L-CDR2
SEQ ID NO: 13
DASIRAT
L-CDR3
SEQ ID NO: 14
QQRYNWLT
IMM20059 HEAVY CHAIN AMINO ACID
SEQ ID NO: 15
EVQLVESGGGLVQPGGSLRLSCAASGFTFSIHSLNWVRQAPGKGLEWVSYI
SSNSTTIYYADSVKGRFTISRDNAKDSLYLQMNSLRDEDTAVYYCARDYYC
TGGTCFFLPDLWGRGALVTVSSASTKGPSVFPLAPSSKSTSGGTAALGCLV
KDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
IMM20059 LIGHT CHAIN AMINO ACID
SEQ ID NO: 16
EIVLTQSPATLSLSPGERATLSCRASQNISNFLAWYQHKPGQAPRLLIYDA
SIRATGIPARFSGSGSGTDFSLTISSLEPEDFAVYFCQQRYNWLTFGGGTK
VEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPV
TKSFNRGEC
GST-EPN1
SEQ ID NO: 17
MESPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVLD
IRYGVSRIAYSKDFETLKVDFLSKLPEMLKMFEDRLCHKTYLNGDHVTHPD
FMLYDALDVVLYMDPMCLDAFPKLVCFKKRIEAIPQIDKYLKSSKYIAWPL
QGWQATFGGGDHPPKSDLEVLFQGPLETSLYKKAGTMSTSSLRRQMKNIVH
NYSEAEIKVREATSNDPWGPSSSLMSEIADLTYNVVAFSEIMSMIWKRLND
HGKNWRHVYKAMTLMEYLIKTGSERVSQQCKENMYAVQTLKDFQYVDRDGK
DQGVNVREKAKQLVALLRDEDRLREERAHALKTKEKLAQTATASSAAVGSG
PPPEAEQAWPQSSGEEELQLQLALAMSKEEADQEERIRRGDDLRLQMAIEE
SKRETGGKEESSLMDLADVFTAPAPAPTTDPWGGPAPMAAAVPTAAPTSDP
WGGPPVPPAADPWGGPAPTPASGDPWRPAAPAGPSVDPWGGTPAPAAGEGP
TPDPWGSSDGGVPVSGPSASDPWTPAPAFSDPWGGSPAKPSTNGTTAGGFD
TEPDEFSDFDRLRTALPTSGSSAGELELLAGEVPARSPGAFDMSGVRGSLA
EAVGSPPPAATPTPTPPTRKTPESFLGPNAALVDLDSLVSRPGPTPPGAKA
SNPFLPGGGPATGPSVTNPFQPAPPATLTLNQLRLSPVPPVPGAPPTYISP
LGGGPGLPPMMPPGPPAPNTNPFLL
EPN1
SEQ ID NO: 18
MSTSSLRRQMKNIVHNYSEAEIKVREATSNDPWGPSSSLMSEIADLTYNVV
AFSEIMSMIWKRLNDHGKNWRHVYKAMTLMEYLIKTGSERVSQQCKENMYA
VQTLKDFQYVDRDGKDQGVNVREKAKQLVALLRDEDRLREERAHALKTKEK
LAQTATASSAAVGSGPPPEAEQAWPQSSGEEELQLQLALAMSKEEADQEER
IRRGDDLRLQMAIEESKRETGGKEESSLMDLADVFTAPAPAPTTDPWGGPA
PMAAAVPTAAPTSDPWGGPPVPPAADPWGGPAPTPASGDPWRPAAPAGPSV
DPWGGTPAPAAGEGPTPDPWGSSDGGVPVSGPSASDPWTPAPAFSDPWGGS
PAKPSTNGTTAGGFDTEPDEFSDFDRLRTALPTSGSSAGELELLAGEVPAR
SPGAFDMSGVRGSLAEAVGSPPPAATPTPTPPTRKTPESFLGPNAALVDLD
SLVSRPGPTPPGAKASNPFLPGGGPATGPSVTNPFQPAPPATLTLNQLRLS
PVPPVPGAPPTYISPLGGGPGLPPMMPPGPPAPNTNPFLL
IMM20059 (aa 44-72)
SEQ ID NO: 19
GLEWVSYISSNSTTIYYADSVKGRFTISR
IMM20059 (aa 92-102)
SEQ ID NO: 20
YNWLTFGGGTK
IMM20059 (aa 44-67)
SEQ ID NO: 21
GLEWVSYISSNSTTIYYADSVKGR
IMM20059 (aa 55-61)
SEQ ID NO: 22
ATGIPAR
IMM20059 (aa 20-38)
SEQ ID NO: 23
LSCAASGFTFSIHSLNWVR
IMM20059 (aa 19-24)
SEQ ID NO: 24
ATLSCR
IMM20059 (aa 30-36)
SEQ ID NO: 25
SIHSLNW
EPN1 (aa 98-107)
SEQ ID NO: 26
ENMYAVQTLK
EPN1 (aa 108-114)
SEQ ID NO: 27
DFQYVDR
EPN1 (aa 108-117)
SEQ ID NO: 28
DFQYVDRDGK
EPN1 (aa 115-126)
SEQ ID NO: 29
DGKDQGVNVREK
EPN1 (aa 118-126)
SEQ ID NO: 30
DQGVNVREK
EPN1 (aa 127-139)
SEQ ID NO: 31
AKQLVALLRDEDR
EPN1 (aa 135-154)
SEQ ID NO: 32
RDEDRLREERAHALKTKEKL

What is claimed is:

1. An isolated antibody, or antigen-binding fragment thereof, that binds the protein, Epsin 1, comprising a variable heavy chain (V_{H}) and a variable light chain (V_{L}), wherein:
A) the VHC comprises an amino acid that shares at least 90% homology with the amino acid sequence corresponding to SEQ ID NO: 2; and
B) The VLC comprises an amino acid that shares at least 90% homology with the amino acid sequence corresponding to SEQ ID NO: 4.

2. The antibody, or antigen-binding fragment of claim 1, comprising at least one of the following complementary determining region (CDR) amino acid sequences:
CDR-H1, corresponding to SEQ ID NO: 9;
CDR-H2, corresponding to SEQ ID NO: 10;
CDR-H3, corresponding to SEQ ID NO: 11;
CDR-L1, corresponding to SEQ ID NO: 12;
CDR-L2 corresponding to SEQ ID NO: 13; and
CDR-L3 corresponding to SEQ ID NO: 14.

3. The antibody, or antigen-binding fragment of claim 1 or 2, wherein the antibody, or antigen-binding fragment, binds to Epsin-1 (EPN1).

4. The antibody, or antigen-binding fragment of claim 3, wherein the K_{D} between the antibody or antigen-binding fragment and EPN1 is 50 nM or less.

5. The antibody, or antigen-binding fragment of claim 4, wherein the K_{D} is 1 nM or less.

6. The antibody or antigen-binding fragment of any one of claims 1-5, wherein the antibody or fragment, is internalized by a cell, after binding to the antigen expressed on the surface of the cell.

7. The antigen-binding fragment of any one of claims 1-6, wherein the antigen-binding fragment is an isolated variable heavy (V_{H}) single domain monoclonal antibody.

8. The antigen-binding fragment of any one of claims 1-6, wherein the antigen-binding fragment is a single chain (sc)Fv-Fc fragment.

9. The antibody or antigen-binding fragment of claim 7 or 8, wherein the antibody or antigen-binding fragment comprises a CH3 scaffold, comprising at least one modification of the wild-type amino acid sequence of the CH3 domain derived from an immunoglobulin Fc region.

10. The antigen-binding fragment of any one of claims 1-9, wherein the isolated antigen-binding fragment comprises an Fv, scFv, Fab, F(ab′)2, or Fab′ fragment, diabody, or any fragment whose half-life may have been increased.

11. The antibody or antigen-binding fragment of any one of claims 1-10, wherein the antibody or fragment is monoclonal.

12. The antibody or antigen-binding fragment of claim 11, wherein the antibody or antigen-binding fragment is human or humanized

13. The antibody or antigen-binding fragment of claim 12, wherein the antibody or antigen-binding fragment is bi-specific.

14. An isolated immunoconjugate comprising the antibody or antigen-binding fragment of claim 13, and an effector molecule.

15. The isolated immunoconjugate of claim 14, wherein the effector molecule is a drug.

16. The isolated immunoconjugate of claim 14, wherein the effector molecule is a toxin.

17. The isolated immunoconjugate of claim 14, wherein the effector molecule is a radioactive drug.

18. A composition comprising a therapeutically effective amount of an isolated antibody or antigen-binding fragment, or isolated immunoconjugate according to any one of the preceding claims, in a pharmaceutically acceptable carrier.

19. The antibody or antigen-binding fragment of any one of claims 1-16, wherein the antibody or antigen-binding fragment is labeled.

20. The antibody or antigen-binding fragment of claim 18, wherein the label is a fluorescent, enzymatic, or radioactive label.

21. A method of treating a subject suffering from at least one type of cancer, comprising selecting the subject suffering from the cancer, wherein cells of the cancer expresses EPN1, and administering to the subject, a therapeutically effective amount of the composition of claim 18, thereby treating the cancer in the subject.

22. A method of inhibiting tumor growth or metastasis, comprising selecting a subject suffering from at least one type of cancer, comprising selecting the subject suffering from the cancer, wherein cells of the cancer expresses EPN1, and administering to the subject, a therapeutically effective amount of the composition of claim 18, thereby inhibiting tumor growth or metastasis.

23. The method according to claim 21 or 22, wherein the type of cancer is a lung carcinoma, melanoma or hepatocellular carcinoma.

24. An isolated nucleic acid molecule encoding a V_{H} according to claim 1.

25. An isolated nucleic acid molecule encoding the V_{L} according to claim 1.

26. The isolated nucleic acid molecule of claim 23, operably linked to a promoter.

27. The isolated nucleic acid molecule of claim 24, operably linked to a promoter.

28. An expression vector comprising the isolated nucleic acid molecule of claim 29.

29. An isolated expression vector comprising the isolated nucleic acid molecule of at least one of claims 27 and 28.

30. An isolated host cell transformed with the nucleic acid molecule or expression vector of claim 29.


SEQUENCE LISTING (32 sequences)

SEQ ID NO: 1 - DNA, 354 bp, Homo sapiens
gactctcctgtgcagcctctggattcaccttcagtatccatagcctgaattgggtccgccaggctccagggaagggactggagtgggtttcgtatattagtagtaacagtactaccatatattacgcagactctgtgaagggccgattcaccatctccagagacaatgccaaggactccctgtatctgcaaatgaacagcctcagagacgaggacacggctgtatattactgtgcgagagactactactgtactggtggtacctgcttctttcttcctgacctctggggccggggagccctggtcaccgtctcctcagcctccaccaagggcccatcggtcttccccctggcgc

SEQ ID NO: 2 - protein, 118 aa, Homo sapiens
LSCAASGFTFSIHSLNWVRQAPGKGLEWVSYISSNSTTIYYADSVKGRFTISRDNAKDSLYLQMNSLRDEDTAVYYCARDYYCTGGTCFFLPDLWGRGALVTVSSASTKKGPSVFPLA

SEQ ID NO: 3 - DNA, 407 bp, Homo sapiens
acaggcgcgcactccgaggtgcagctggtggagtctgggggaggcttggtacagcctggggggtccctgagactctcctgtgcagcctctggattcaccttcagtatccatagcctgaattgggtccgccaggctccagggaagggactggagtgggtttcgtatattagtagtaacagtactaccatatattacgcagactctgtgaagggccgattcaccatctccagagacaatgccaaggactccctgtatctgcaaatgaacagcctcagagacgaggacacggctgtatattactgtgcgagagactactactgtactggtggtacctgcttctttcttcctgacctctggggccggggagccctggtcaccgtctcctcagcctccaccaagggcccatc

SEQ ID NO: 4 - protein, 135 aa, Homo sapiens
EVQLVESGGGLVQPGGSLRLSCAASGFTFSIHSLNWVRQAPGKGLEWVSYISSNSTTIYYADSVKGRFTISRDNAKDSLYLQMNSLRDEDTAVYYCARDYYCTGGTCFFLPDLWGRGALVTVSSASTKGPSVFPL

SEQ ID NO: 5 - DNA, 299 bp, Homo sapiens
aagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaacgaactgtggctgcaccatctgtcttcatct

SEQ ID NO: 6 - protein, 99 aa, Homo sapiens
RATLSCRASQNISNFLAWYQHKPGQAPRLLIYDASIRATGIPARFSGSGSGTDFSLTISSLEPEDFAVYFCQQRYNWLTFGGGTKVEIKRTVAAPSVFI

SEQ ID NO: 7 - DNA, 346 bp, Homo sapiens
tcagatacctccggagaaattgtgttgacacagtctccagccaccctgtctttgtctccaggggaaagagccaccctctcctgcagggccagtcagaatatcagcaacttcttagcctggtaccaacacaaacctggccaggctcccaggctcctcatctatgatgcatccatcagggccactggcatcccagccaggttcagtggcagtgggtctgggacagacttcagtctcaccatcagcagcctggagcctgaagattttgcagtttatttctgtcagcagcgttacaactggctcactttcggcggagggaccaaggtagagatcaaacgaactgtggctg

SEQ ID NO: 8 - protein, 110 aa, Homo sapiens
EIVLTQSPATLSLSPGERATLSCRASQNISNFLAWYQHKPGQAPRLLIYDASIRATGIPARFSGSGSGTDFSLTISSLEPEDFAVYFCQQRYNWLTFGGGTKVEIKRTVA

SEQ ID NO: 9 - protein, 6 aa, Homo sapiens
SIHSLN

SEQ ID NO: 10 - protein, 17 aa, Homo sapiens
YISSNSTTIYYADSVKG

SEQ ID NO: 11 - protein, 15 aa, Homo sapiens
DYYCTGGTCFFLPDL

SEQ ID NO: 12 - protein, 11 aa, Homo sapiens
RASQNISNFLA

SEQ ID NO: 13 - protein, 7 aa, Homo sapiens
DASIRAT

SEQ ID NO: 14 - protein, 8 aa, Homo sapiens
QQRYNWLT

SEQ ID NO: 15 - protein, 454 aa, Homo sapiens
EVQLVESGGGLVQPGGSLRLSCAASGFTFSIHSLNWVRQAPGKGLEWVSYISSNSTTIYYADSVKGRFTISRDNAKDSLYLQMNSLRDEDTAVYYCARDYYCTGGTCFFLPDLWGRGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 213 aa, Homo sapiens
EIVLTQSPATLSLSPGERATLSCRASQNISNFLAWYQHKPGQAPRLLIYDASIRATGIPARFSGSGSGTDFSLTISSLEPEDFAVYFCQQRYNWLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 790 aa, Artificial
  GST-EPN1_fusion
MESPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLETSLYKKAGTMSTSSLRRQMKNIVHNYSEAEIKVREATSNDPWGPSSSLMSEIADLTYNVVAFSEIMSMIWKRLNDHGKNWRHVYKAMTLMEYLIKTGSERVSQQCKENMYAVQTLKDFQYVDRDGKDQGVNVREKAKQLVALLRDEDRLREERAHALKTKEKLAQTATASSAAVGSGPPPEAEQAWPQSSGEEELQLQLALAMSKEEADQEERIRRGDDLRLQMAIEESKRETGGKEESSLMDLADVFTAPAPAPTTDPWGGPAPMAAAVPTAAPTSDPWGGPPVPPAADPWGGPAPTPASGDPWRPAAPAGPSVDPWGGTPAPAAGEGPTPDPWGSSDGGVPVSGPSASDPWTPAPAFSDPWGGSPAKPSTNGTTAGGFDTEPDEFSDFDRLRTALPTSGSSAGELELLAGEVPARSPGAFDMSGVRGSLAEAVGSPPPAATPTPTPPTRKTPESFLGPNAALVDLDSLVSRPGPTPPGAKASNPFLPGGGPATGPSVTNPFQPAPPATLTLNQLRLSPVPPVPGAPPTYISPLGGGPGLPPMMPPGPPAPNTNPFLL

SEQ ID NO: 18 - protein, 550 aa, Homo sapiens
MSTSSLRRQMKNIVHNYSEAEIKVREATSNDPWGPSSSLMSEIADLTYNVVAFSEIMSMIWKRLNDHGKNWRHVYKAMTLMEYLIKTGSERVSQQCKENMYAVQTLKDFQYVDRDGKDQGVNVREKAKQLVALLRDEDRLREERAHALKTKEKLAQTATASSAAVGSGPPPEAEQAWPQSSGEEELQLQLALAMSKEEADQEERIRRGDDLRLQMAIEESKRETGGKEESSLMDLADVFTAPAPAPTTDPWGGPAPMAAAVPTAAPTSDPWGGPPVPPAADPWGGPAPTPASGDPWRPAAPAGPSVDPWGGTPAPAAGEGPTPDPWGSSDGGVPVSGPSASDPWTPAPAFSDPWGGSPAKPSTNGTTAGGFDTEPDEFSDFDRLRTALPTSGSSAGELELLAGEVPARSPGAFDMSGVRGSLAEAVGSPPPAATPTPTPPTRKTPESFLGPNAALVDLDSLVSRPGPTPPGAKASNPFLPGGGPATGPSVTNPFQPAPPATLTLNQLRLSPVPPVPGAPPTYISPLGGGPGLPPMMPPGPPAPNTNPFLL

SEQ ID NO: 19 - protein, 29 aa, Homo sapiens
GLEWVSYISSNSTTIYYADSVKGRFTISR

SEQ ID NO: 20 - protein, 11 aa, Homo sapiens
YNWLTFGGGTK

SEQ ID NO: 21 - protein, 24 aa, Homo sapiens
GLEWVSYISSNSTTIYYADSVKGR

SEQ ID NO: 22 - protein, 7 aa, Homo sapiens
ATGIPAR

SEQ ID NO: 23 - protein, 19 aa, Homo sapiens
LSCAASGFTFSIHSLNWVR

SEQ ID NO: 24 - protein, 6 aa, Homo sapiens
ATLSCR

SEQ ID NO: 25 - protein, 7 aa, Homo sapiens
SIHSLNW

SEQ ID NO: 26 - protein, 10 aa, Homo sapiens
ENMYAVQTLK

SEQ ID NO: 27 - protein, 7 aa, Homo sapiens
DFQYVDR

SEQ ID NO: 28 - protein, 10 aa, Homo sapiens
DFQYVDRDGK

SEQ ID NO: 29 - protein, 12 aa, Homo sapiens
DGKDQGVNVREK

SEQ ID NO: 30 - protein, 9 aa, Homo sapiens
DQGVNVREK

SEQ ID NO: 31 - protein, 13 aa, Homo sapiens
AKQLVALLRDEDR

SEQ ID NO: 32 - protein, 20 aa, Homo sapiens
RDEDRLREERAHALKTKEKL